(19) United States
(12) Patent Application Publication
(10) Pub. No.: US 2022/0354898 A1
(43) Pub. Date: Nov. 10, 2022

(54) INDUCTION OF TUMOR VASCULAR NECROSIS UTILIZING FIBROBLASTS

(71) Applicant: FibroBiologics, Houston, TX (US)

(72) Inventors: Thomas ICHIM, San Diego, CA (US); Pete O'HEERON, Houston, TX (US)

(21) Appl. No.: 17/738,584

(22) Filed: May 6, 2022

Related U.S. Application Data
(60) Provisional application No. 63/184,960, filed on May 6, 2021.

(51) Int. Cl. A61K 35/33; A61K 31/395; C12N 5/0735; C12N 5/077

(52) U.S. Cl. CPC A61K 35/33; A61K 31/395; C12N 5/0606; C12N 5/0656; C12N 2501/105; C12N 2501/165; C12N 2501/11; C12N 2501/12; C12N 2510/00; C12N 2506/1307; C12N 2506/03

(57) ABSTRACT

Embodiments of the disclosure concern methods and compositions related to cancer treatment for an individual utilizing recombinant fibroblast cells that comprise one or more activities that are endothelial cell-like. The cells are delivered to a tumor microenvironment following which their death results in destabilization of the tumor vasculature. In particular embodiments, the fibroblast cells recombinantly express one or more of ETV2, FOXC2, and FLI1.

### CROSS-REFERENCE TO RELATED APPLICATIONS

This application claims priority to U.S. Provisional Patent Application Ser. No. 63/184,960, filed May 6, 2021, which is incorporated by reference herein in its entirety.

### TECHNICAL FIELD

Embodiments of the disclosure concern at least the fields of cell biology, molecular biology, and medicine.

### BACKGROUND

The use of the immune system for killing cancer is an area of active investigation. Immunological control of neoplasia is suggested by: A) Evidence of longer survival of patients with a variety of cancers who possess a high population of tumor infiltrating lymphocytes [1-3]; B) The fact that immune suppressed patients develop cancer at a much higher frequency in comparison to non-immune suppressed individuals [4, 5]; and C) In some very particular situations immunotherapy of cancer is clinically effective [6]. While cancer immunotherapy offers the possibility of inducing remission and control of both the primary tumor mass, as well as micrometastasis, several drawbacks exist. The most significant one is that in many situations immunotherapy is either not feasible, or associated with a variety of toxicities. Various types of immunotherapies for cancer have been attempted, including: a) systemic cytokine administration; b) gene therapy; c) allogeneic vaccines; d) autologous vaccine; e) heat shock protein vaccines; f) dendritic cell vaccines; g) tumor infiltrating lymphocytes; h) administration of T cells in a lymphodepleted environment; and i) nutritional interventions. Although each of the approaches contains significant advantages and drawbacks, none of them simultaneously meet the criteria of reproducible efficacy, availability to the mass population, or tumor selectivity/specificity.

The limitations of many immunotherapeutic approaches to cancer is that tumor antigens are either not clearly defined, or in situations where they are defined, the tumor either mutates to lose expression of such antigens, or the antigen-specific vaccine is only applicable to patients with a certain major histocompatibility complex haplotype. The circumvention of this problem has been attempted using autologous vaccines, however in many cases this is an expensive and difficult procedure.

### BRIEF SUMMARY

The present disclosure is directed to methods and compositions that directly or indirectly are therapeutic to a recipient individual. In particular embodiments, the therapy comprises cells, including modified cells, that are provided to an individual in need thereof. In particular embodiments, the cells are modified by the hand of man prior to delivery to the individual. In specific embodiments, fibroblasts are modified and a therapeutically effective amount of the modified cells are administered to an individual with a medical condition, such as cancer.

In particular embodiments, fibroblasts are exposed to one or more agents and/or conditions that results in the fibroblasts behaving more endothelial cell-like in activity. In certain embodiments, fibroblasts are exposed to one or more gene products following which they act more like endothelial cells than in the absence of such exposure. In various embodiments, fibroblasts are utilized as a means of generating endothelial cells. The “artificial” endothelial cells produced by methods of the disclosure enhance the ability of the cells to enter the tumor micro environment that then results in death of tumor cells. In various embodiments, once the modified fibroblasts enter the tumor microenvironment, they kill themselves by a suicide or death-inducing gene that causes destabilization of the tumor vasculature. In some embodiments, another therapy of any kind that otherwise would be less effective because of conditions in the tumor microenvironment may then be administered to the individual.

In various embodiments, fibroblasts express one or more recombinant genes (as opposed to genes that are endogenous to the fibroblasts) that facilitate their activity to be more like endothelial cells. In specific embodiments, the fibroblasts are modified to express one or more of ETV2, FOXC2, and FLI1 that results in the modified fibroblasts to exhibit one or more properties of endothelial cells and/or vascular channels. In embodiments wherein two or more of ETV2, FOXC2, and FLI1, the two or more genes may or may not be expressed from the same polynucleotide, such as a transfected vector of any kind.

In various embodiments, the disclosure provides one or more different ways of making “artificial” tumor endothelial cells to act as an immunogenic composition, such as a “vaccine,” in order to induce immunity to cancer blood vessels. In specific embodiments, transarterial chemoembolization (TACE) is utilized as one means of stimulating immunity to cancer endothelium.

Embodiments of the disclosure include methods of inducing cell death of tumor cells in an individual, comprising the step of administering to the individual a therapeutically effective amount of a plurality of modified fibroblasts, wherein: (I) the fibroblasts express recombinant: (a) one or more endothelial-inducing genes and/or one or more vascular channel-inducing genes (or one or more factors that upregulate same); and (b) one or more suicide or death-inducing genes (or one or more factors that upregulate same); and (c) optionally, one or more immune stimulatory genes; and/or (II) the fibroblasts are cultured in endothelial progenitor cell conditioned media. In specific embodiments, the fibroblasts express recombinant ETV2, FOXC2, and/or FLI1. In certain cases, the fibroblasts express recombinant ETV2 and are cultured with media that comprises an effective amount of one or more of VEGF, EGF, HGF, and IGF-1. In certain cases, the fibroblasts express recombinant FOXC2 and are cultured with media that comprises an effective amount of one or more of VEGF, EGF, HGF, and IGF-1. In certain cases, the fibroblasts express recombinant FLI1 and are cultured with media that comprises an effective amount of one or more of VEGF, EGF, HGF, and IGF-1. In particular embodiments, endothelial cell progenitor cell conditioned media is generated from pluripotent stem cells differentiated into endothelial progenitor cells. The pluripotent stem cells may be embryonic stem cells, inducible pluripotent stem cells, somatic nuclear transfer derived stem cells, parthenogenically derived stem cells, or differentiated into endothelial progenitor cells by transfection of ETV2, FOXC2, and FLI1. In various embodiments, the fibroblasts are transfected with one or more thrombosis-associated genes, wherein said gene is upregulated in response to hypoxia. The thrombosis-associated gene may be tissue factor or an inhibitor of Protein C.

In particular embodiments, the fibroblasts are transfected with one or more immune stimulatory genes and may be inducible by the presence of hypoxia. Induction of the immune stimulatory gene may be performed by placing the gene under control of the HIF-1 alpha transcription factor. The immune stimulatory gene may be associated with antigen presentation, such as an allogeneic MHC molecule. The gene associated with antigen presentation may be a xenogeneic MHC molecule. In specific embodiments, the gene associated with antigen presentation may be one or more of the HLA B7 molecule, CD80, CD86, and CD40. The immune stimulatory gene may be interleukin-12.

In various embodiments, the fibroblasts are selected for expression of one or more of CXCR4, CD73, CD74, CD206, and interleukin-3 receptor. The fibroblasts may be either allogeneic, syngeneic, or xenogeneic to any recipient.

In certain embodiments, there are methods for inducing immunogenic cell death of tumor endothelial cells in an individual, comprising the steps of: a) transfecting a fibroblast population with one or more endothelial cell-inducing genes and/or one or more vascular channel-inducing genes; c) transfecting said fibroblasts with one or more suicide or death-inducing genes; d) optionally transfecting said fibroblasts with one or more immune stimulatory genes; and e) administering said fibroblasts into an individual with cancer. The fibroblasts may be obtained from one or more tissues selected from the group consisting of a) dermal; b) bone marrow; c) blood; d) mobilized peripheral blood; e) gingiva; f) tonsil; g) placenta; h) Wharton's Jelly; i) hair follicle; j) fallopian tube; k) liver; 1) deciduous tooth; m) vas deferens; n) endometrial; o) menstrual blood; and p) omentum. In specific embodiments, mobilization of peripheral blood is achieved through treatment of a mammal with an effective amount of one or more inhibitors of SDF-1 binding to CXCR4. The inhibitor of SDF-1 binding to CXCR4 may be Plerixafor or BKT140. The mobilization may be induced by exposure to hyperbaric oxygen treatment. Mobilization may be induced by treatment with GM-CSF and/or M-CSF and/or with flt-3 ligand. In various embodiments, the fibroblasts are selected for expression of one or more of CXCR4, CD73, CD74, CD206, and interleukin-3 receptor. The fibroblasts may be either allogeneic, syngeneic, or xenogeneic to any recipient.

In particular embodiments, fibroblasts are cultured in endothelial progenitor cell-conditioned media, such as media generated from pluripotent stem cells differentiated into endothelial progenitor cells. The pluripotent stem cells may be embryonic stem cells, inducible pluripotent stem cells, somatic nuclear transfer derived stem cells, parthenogenically derived stem cells, or differentiated into endothelial progenitor cells by transfection of ETV2, FOXC2, and FLI1. The fibroblasts may be differentiated into endothelial progenitor cells by transfection with ETV2. The fibroblasts may be differentiated into endothelial progenitor cells by transfection with ETV2 and culture in media that comprises VEGF. The fibroblasts may be differentiated into endothelial progenitor cells transfection with ETV2 and culture in media that comprises EGF. The fibroblasts may be differentiated into endothelial progenitor cells by transfection with ETV2 and culture in media that comprises HGF. The fibroblasts may be differentiated into endothelial progenitor cells by transfection with ETV2 and culture in media that comprises IGF-1. The fibroblasts may be differentiated into endothelial progenitor cells by transfection with FOXC2. The fibroblasts may be differentiated into endothelial progenitor cells by transfection with FOXC2 and culture in media that comprises VEGF. The fibroblasts may be differentiated into endothelial progenitor cells by transfection with FOXC2 and culture in media that comprises EGF. The fibroblasts may be differentiated into endothelial progenitor cells by transfection with FOXC2 and culture in media that comprises HGF. The fibroblasts may be differentiated into endothelial progenitor cells by transfection with FOXC2 and culture in media that comprises IGF-1. The fibroblasts may be differentiated into endothelial progenitor cells by transfection with FLI1. The fibroblasts may be differentiated into endothelial progenitor cells by transfection with FLI1 and culture in media that comprises VEGF. The fibroblasts may be differentiated into endothelial progenitor cells by transfection with FLI1 and culture in media that comprises EGF. The fibroblasts may be differentiated into endothelial progenitor cells by transfection with FLI1 and culture in media that comprises HGF. The fibroblasts may be differentiated into endothelial progenitor cells by transfection with FLI1 and culture in media that comprises IGF-1.

In some embodiments, the fibroblasts are transfected with one or more thrombosis inducing genes or molecules, wherein said gene is upregulated in response to hypoxia. The thrombosis associated gene or molecule may be tissue factor or is an inhibitor of Protein C. In some embodiments, the fibroblast is transfected with one or more immune stimulatory genes that may be inducible by the presence of hypoxia. In some cases, induction of the immune stimulatory gene is performed by placing the gene under control of the HIF-1 alpha transcription factor. The immune stimulatory gene may be associated with antigen presentation, including antigen presentation that is an allogeneic MHC molecule. The gene associated with antigen presentation may be a xenogeneic MHC molecule. The gene associated with antigen presentation may be the HLA B7 molecule, CD80, CD86, and/or CD40. The immune stimulatory gene may be interleukin-12.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

### I. Examples of Definitions

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

“Treating” or treatment of a disease or condition refers to executing a protocol, which may include administering one or more drugs to an individual, such as a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease, including breast cancer.

“Marker” and “Biomarker” are used interchangeably to refer to a gene expression product that is differentially present in a samples taken from two different subjects, e.g., from a test subject or patient having (a risk of developing) an ischemic event, compared to a comparable sample taken from a control subject (e.g., a subject not having (a risk of developing) an ischemic event; a normal or healthy subject). Alternatively, the terms refer to a gene expression product that is differentially present in a population of cells relative to another population of cells.

The phrase “differentially present” refers to differences in the quantity or frequency (incidence of occurrence) of a marker present in a sample taken from a test subject as compared to a control subject. For example, a marker can be a gene expression product that is present at an elevated level or at a decreased level in blood samples of a risk subjects compared to samples from control subjects. Alternatively, a marker can be a gene expression product that is detected at a higher frequency or at a lower frequency in samples of blood from risk subjects compared to samples from control subjects.

A gene expression product is “differentially present” between two samples if the amount of the gene expression product in one sample is statistically significantly different from the amount of the gene expression product in the other sample. For example, a gene expression product is differentially present between two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

As used herein, the terms “antibody” and “antibodies” refer to monoclonal antibodies, multispecific antibodies, synthetic antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to a polypeptide antigen. The immunoglobulin molecules of the invention can be of any type (e.g., IgG, IgE, IgM, IgD, IgA and IgY), class (e.g., IgG.sub.1, IgG.sub.2, IgG.sub.3, IgG.sub.4, IgA.sub.1 and IgA.sub.2) or subclass of immunoglobulin molecule.

“Immunoassay” is an assay that uses an antibody to specifically bind an antigen (e.g., a marker). The immunoassay is characterized by the use of specific binding properties of a particular antibody to isolate, target, and/or quantify the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background. The phrase “specifically (or selectively) binds” when referring to an antibody, or “specifically (or selectively) immunoreactive with”, when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

The terms “affecting the expression” and “modulating the expression” of a protein or gene, as used herein, should be understood as regulating, controlling, blocking, inhibiting, stimulating, enhancing, activating, mimicking, bypassing, correcting, removing, and/or substituting said expression, in more general terms, intervening in said expression, for instance by affecting the expression of a gene encoding that protein.

### II. General and Specific Embodiments

Disclosed herein are certain embodiments of methods, means, protocols and compositions of matter useful for induction of tumor regression, such as by inciting vascular necrosis of tumor-associated blood vessels. In one embodiment, the disclosure provides methods of transfecting fibroblasts with one or more genes, such as one or more death-inducing genes or means of upregulating same. In particular embodiments, the genes are transcription factors (including hypoxia-inducible transcription factors) that upregulate one or more death-inducing genes. In one embodiment, fibroblasts are transdifferentiated into endothelial cells, including tumor endothelial cells, wherein the differentiated endothelial cells, or endothelial cell-like cells, initiate a coagulation and/or complement cascade leading to selected necrosis of the tumor vasculature. Examples of death-inducing genes include TNF alpha, TNF beta, FAS ligand and TRAIL.

In some embodiments, the disclosure encompasses the utilization of endothelial targeting using fibroblasts and/or fibroblasts that are differentiated into endothelial cells as a means of augmenting efficacy of cancer antigen-specific vaccines and/or inducing vascular necrosis for a tumor. In one embodiment, the disclosure encompasses administration to an individual of fibroblasts of any kind, including, for example, placentally-derived fibroblasts, that are differentiated into endothelial cells that resemble cancer endothelial cells. Targeting of tumor endothelial cells using vaccines is possible, however generation from a standardized source has not been performed [7-10]. The disclosure provides for embodiments wherein tumors are sensitized to treatment with cancer therapeutics, including at least cancer vaccines. In specific embodiments, the cancer vaccines comprise peptide vaccines, protein vaccines [11], cellular vaccines, and/or endogenous vaccines. Without being bound to theory, the cancer endothelial targeting using fibroblast approaches are capable of specifically inducing inactivation of tumor endothelial-mediated lymphocyte death, thus allowing for cancer killing T cells to specifically enter the tumor and mediate tumor cell death. In particular embodiments, the treatment for which the tumors are sensitized comprises any kind of adoptive cell therapy, including T cells and/or NK cells that are engineered to express a non-endogenous antigen receptor, including a chimeric antigen receptor, a T-cell receptor, and so forth.

In one embodiment, endothelial progenitor cells (EPC) generated from fibroblasts may be used to stimulate immunity to tumor endothelium. The endothelial cells produced by methods encompassed herein, in one embodiment, comprises a population of cells having one or more particular surface markers on each cell or the majority of cells. Embodiments include a cell population comprises cells having the surface marker CD44 [12], cells having the surface marker CD13 [13, 14], cells having the surface marker CD90 [12, 15-20], cells having the surface marker CD105 [13, 16, 21-27], cells having the surface marker ABCG2, cells having the surface marker HLA 1, cells having the surface marker CD34, cells having the surface marker CD133, cells having the surface marker CD117, cells having the surface marker CD135, cells having the surface marker CXCR4, cells having the surface marker c-met, cells having the surface marker CD31, cells having the surface marker CD14, cells having the surface marker Mac-1, cells having the surface marker CD11, cells having the surface marker c-kit cells having the surface marker SH-2, cells having the surface marker VE-Cadherin, cells having the surface marker VEGFR, and//or cells having the surface marker Tie-2s. The EPC may be treated in a manner to mimic the tumor microenvironment; specifically, they may be grown under the acidic conditions in the tumor microenvironment, information of which is incorporated by reference [28-41]. In one embodiment of the disclosure, endothelial progenitor cells, or products thereof, are cultured under conditions in which GCN2 kinase is activated [42, 43], and in specific cases the conditions include culture in the presence of uncharged tRNA [44-47], tryptophan deprivation [48-50], arginine deprivation [51-56], asparagine deprivation [57-61], and/or glutamine deprivation [62, 63].

In particular embodiments, generation of endothelial cells may be produced from fibroblasts after the fibroblasts have been transfected with one or more various immune modulatory genes, including combinations of genes. In some embodiment, genes associated with immune modulation are transfected into the cells. In specific embodiments, the genes include one or more interleukins, one or more HLA molecules, one or more costimulatory molecules, and/or one or more adhesion molecules.

In one embodiment, fibroblasts are transfected with one or more cytokine genes, such as interleukin-12, subsequently induced to differentiate into endothelial cells, and then the endothelial cells are administered either systemically or locally in the tumor. Administration of fibroblast-derived endothelial cells allows for induction of immunity to tumor endothelial cells, in specific embodiments.

In another embodiment, one or more immunologically active components are transfected under control of hypoxia-inducible elements, and the endothelial cells derived from fibroblasts are injected intratumorally in order to induce immune response against hypoxic elements.

The disclosure utilizes compositions and methods for reprogramming somatic cells into vasculogenic cells and/or endothelial cells both in vitro and in vivo for use in targeting cancer cells. One embodiment includes a polynucleotide comprising two or more nucleic acid sequences encoding proteins selected from the group consisting of ETV2, FOXC2, and FLI1 for generation of a cancer vaccine.

In some embodiments, fibroblasts are reprogrammed into cancer-therapeutic endothelial cells as previously disclosed in U.S. Publication 20200115425, which is incorporated by reference herein. In one embodiment, EPCs refer to endothelial colony-forming cells (ECFCs) and their progenitor cell capacities were characterized as described (Wu, Y et al., J Thromb Haemost, 2010; 8:185-193; Wang, H et al., Circulation research, 2004; 94:843 and Stellos, K et al., Eur Heart J., 2009; 30:584-593). Briefly, human blood was collected from healthy volunteer donors. All volunteers had no risk factors of CVD including hypertension, diabetes, smoking, positive family history of premature CVD and hypercholesterolemia, and were all free of wounds, ulcers, retinopathy, recent surgery, inflammatory, malignant diseases, and medications that may influence EPC kinetics. After dilution with HBSS (1:1), blood was overlaid onto Histopaque 1077 (Sigma-Aldrich Co. LLC, St. Louis, Mo.) in the ratio of 1:1 and centrifuged at 740 g for 30 minutes at room temperature. Buffy coat MNCs were collected and centrifuged at 700 g for 10 minutes at room temperature. MNCs were cultured in collagen type I (BD Bioscience, San Diego) (50 m/ml)-coated dishes with EBM2 basal medium (Lonza Inc., Allendale, N.J.) plus standard EGM-2 SingleQuotes (Lonza Inc., Allendale, N.J.) that includes 2% fetal bovine serum (FBS), EGF (20 ng/ml), hydrocortisone (1 .mu·g/ml), bovine brain extract (12 .mu·g/ml), gentamycin (50 m/ml), amphotericin B (50 ng/ml), and epidermal growth factor (10 ng/ml). Colonies appeared between 5 and 22 days of culture were identified as a well-circumscribed monolayer of cobblestone-appearing cells. ECFCs with endothelial lineage markers expression, robust proliferative potential, colony-forming, and vessel-forming activity in vitro are defined as EPCs as described (Wang, H et al., Circulation research, 2004; 94:843 and Stellos, K et al., Eur Heart J., 2009; 30:584-593). Passage 4 to 6 EPCs were used for experiments. For a brief characterization, endothelial phagocytosis function was confirmed by incubating EPC in 4-well chamber slide with 1,1-dioctadecyl-3,3,3,3-tetramethylindocarbocyanine (DiI)-labeled acetylated low-density lipoprotein (acLDL) (Biomedical Technologies, Inc., Stoughton, Mass.) (5 m/ml) at 37.degree. C. for 1 h, washed 3 times for 15 min in PBS, and then fixed with 2% paraformaldehyde for 10 min. Cells were then incubated with FITC conjugated UEA-1 (Ulex europaeus agglutinin) (10 m/ml) (Sigma-Aldrich Corporation, St. Louis, Mo.) for 1 h at room temperature, which is capable of binding with glycoproteins on the cell membrane to allow visualization of the entire cell. Cell integrity was examined by nuclear staining with DAPI (100 ng/ml). After staining, cells are imaged with high-power fields under an inverted fluorescent microscope (Axiovert 200, Carl Zeiss, Thornwood, N.Y.) at 200.times. magnification and quantified using Image J software.

In particular embodiments, EPC may be identified by means by selecting for cells expressing one or more certain genes. In specific embodiments, the cell expresses one or more genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1). In specific embodiments, the EPC may be purified from a variety of sources, including peripheral blood, placental cells, cord blood, umbilical cord, adipose tissue and/or bone marrow.

In another embodiment, EPC are characterized by expression of at least one gene and in specific embodiments at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1). In a specific embodiment, the EPC are characterized by expression of one or at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1). In specific embodiments, a step of increasing the number of activated endothelial progenitor cells comprises increasing in the endothelial progenitor cells in the blood of a subject the expression of at least one gene and even more preferably at least 2, 3, 4, 5, 10, 15, 20, 25, 30, 35, or all genes selected from the group consisting of ADORA1, ADORA2A, ADORA2B, ADORA3, AGTRL1 (APLNR), AMPH, APLN, CCBE1, CDC42, CGNL1, CREBBP, CRIP1, CRIP2, CRIP3, CYB5B, DLL4, DUSP5, EEA1, egr-1, ELK1, ELK3, ELK4 (SAP1), EP300, ERG1 (KCNH2), ETS1, ETS2, EXOC3L, FGD1, FGD2, FGD3, FGD4, FGD5, FLT1, FST, GATA6, GRRP1, HO-1 (HMOX1), HO-2 (HMOX2), IFNG, IL1A, IL1B, LAMA4, Lamb1-1, LGMN, MMP3, Nos2, PAI1, PHD1, PLVAP, RAB5a, RIN3, ROCK2, SOX18, SOX7, SRF, STAB1, STAB2, STUB1, TFEC, THBS1, THBS2, THBS3, THBS4, THBS5, THSD1, TNFAIP8, and XLKD1 (LYVE1), and in some cases at least one gene or at least 2, 3, 4, 5, 10, 15, 20, 25, 30 or all genes selected from the group consisting of ADORA2A, AGTRL1 (APLNR), APLN, CCBE1, CGNL1, CRIP2, CYB5B, DLL4, DUSP5, ELK3, ERG1 (KCNH2), ETS1, ETS2, EXOC3L, FGD5, GRRP1, HO-1 (HMOX1), HO-2 (HMOX2), LAMA4, Lamb1-1, LGMN, PLVAP, RIN3, ROCK2, SOX7, SOX18, STAB1, STAB2, STUB1, TFEC, THSD1, TNFAIP8, and XLKD1 (LYVE1).

The generation of EPC and EPC-derived endothelial cells may occur through culture of EPC or EPC-derived endothelial cells in conditions that resemble the tumor microenvironment. One such condition is exposure to ionic concentrations that resemble the tumor microenvironment. It is known that tumors contain areas of cellular necrosis, which are associated with poor survival in a variety of cancers. A study showed that necrosis releases intracellular potassium ions into the extracellular fluid of mouse and human tumors, causing profound suppression of T cell effector function. Elevation of the extracellular potassium concentration ([K+]e) impairs T cell receptor (TCR)-driven Akt-mTOR phosphorylation and effector programs. Potassium-mediated suppression of Akt-mTOR signaling and T cell function is dependent upon the activity of the serine/threonine phosphatase PP2A. Although the suppressive effect mediated by elevated [K+]e is independent of changes in plasma membrane potential (Vm), it requires an increase in intracellular potassium ([K+]i). Accordingly, augmenting potassium efflux in tumor-specific T cells by overexpressing the potassium channel Kv1.3 lowers [K+]i and improves effector functions in vitro and in vivo and enhances tumor clearance and survival in melanoma-bearing mice. In one embodiment, there is use of culture conditions similar to those associated with necrotic tissue as a means of modifying EPC and EPC-derived endothelial cells to render the cells similar to tumor endothelial cells [64]. In some embodiments, EPC or EPC-derived endothelial cells are cultured under conditions of free adenosine similar to those found in tumor cells. Numerous publications report concentrations found in tumors and several are incorporated by reference herein [65-74]. In one embodiment, EPC or endothelial cells derived thereof are cultured with one or more enzymes known to induce production of adenosine locally in a manner similar to that found in the tumor microenvironment. Enzymes or ectoenzymes useful for the practice of methods of the disclosure include CD39 [75-78] and/or CD73 [79], which are described in the associated references and incorporated herein [80].

The tumor endothelium acts as a protective barrier to the immune system from attacking the cancer. In certain embodiments, tumor endothelial targeting vaccines are used to reduce or substantially abrogate the ability of the tumor endothelium to protect the tumor from infiltrating immune cells.

One example of how the cancer endothelium protects the tumor is through expression of FasL. FasL was discovered and cloned by Suda et al in 1993 as a member of the tumor necrosis factor family [81], which was subsequently showed to induce apoptosis in various cells expressing Fas, such as T lymphocytes [82]. It is known that FasL and Fas, play a key role in the regulation of apoptosis in the immune system. FasL acts as a cytotoxic effector molecule to Fas-expressing malignant tumor cells; however, it has recently been suggested that FasL also acts as a possible mediator of tumor immune privilege. In a recent study, FasL expression in glioblastoma associated endothelial cells were examined by Western blotting and immunohistochemistry. In addition, quantitative analysis of T-cell infiltration in these tumors was performed. FasL expression was seen in all cell lines and in 9 of 14 specimens by Western blotting and immunohistochemistry. The distribution of FasL was recognized in the tumor vascular. Both types of FasL expression were associated with a significant reduction (p<0.05) in T-cell infiltration when compared with FasL-negative areas within the same tumor or FasL-negative specimens. Because T-cell apoptosis could be induced by FasL-expressing tumor endothelial cells, the authors considered that apoptosis induction by FasL expressed on tumor cells and/or vascular endothelium might be one mechanism for T-cell depletion in astrocytic tumor tissues [83]. Thus it appears that prevention of T cells from entering tumors is mediated in part by the barrier posed by the blood vessel containing death ligands. The importance of FasL maintaining immune privilege has been observed in physiological situations. For example, immune privilege of the eye [84-89], the nucleus pulposus of the intravertebral disc [90, 91], the testis [92-106], the blood brain barrier [107], and the placenta [108-111], is associated with expression of FasL. In another study, investigators sought to determine T cell presence in TIL, and the ratio of CD8+ and CD4+ T cell subsets in particular, can correlate with tumor prognosis in some tumors, although the significance of such infiltration into glioma is controversial. However, gliomas represent a lower extreme in their extent of T cell infiltration, and are thus useful in assessing factors that can decrease T cell presence within tumor tissue. Fas ligand, a pro-apoptotic cell surface protein, may play a key role in reduction of T cells in tumor tissue. To assess the level of FasL expression on brain tumor endothelium and to correlate this with relative levels of CD4+ and CD8+ T cell subsets in TLL from brain tumors. CD3+, CD4+, and CD8+ cells were quantified in fresh TIL by flow cytometry. Paraffin embedded sections of tumors, including meningiomas and gliomas as well as extracranial malignancies, underwent immunohistochemical staining for FasL and Von-Willebrand's factor (Factor VIII) to determine expression levels of endothelial FasL. FasL expression was high in aggressive intracranial malignancies compared to more indolent neoplasms, and correlated inversely with CD8+/CD4+ TIL ratios in all tumor classes combined (ANOVA, p<0.05). Low levels of T cells within TIL, as well as low CD8+/CD4+ TIL ratios appear to be a property of parenchymal tumor presence. Together with the inverse correlation seen between FasL expression and CD8+/CD4+ TIL ratios, the high levels of endothelial FasL expression in gliomas suggests that FasL decreases T cell presence in brain tumors in a subset-selective manner, thus contributing to glioma immune privilege [112].

Patients in which infiltration of T cells and NK cells (tumor infiltrating lymphocytes (TILs)) is observed possess a better prognosis as compared to patients without tumor infiltrating lymphocytes. Thus in one embodiment, endothelial-targeting vaccines are utilized as a means of augmenting ability of lymphocytes to enter the tumor. TILs have been noticed in a variety of tumors and are correlated with a favorable prognosis in certain cancers including liver carcinoma [113], melanoma [114, 115], bladder cancer [116], colorectal cancer [117], and ovarian cancer [118, 119]. It is the belief of many tumor immunologists that TILs infiltrate tumors to induce their eradication, however, this does not occur in vivo because tumor-secreted immunosuppressive factors inhibit immune activation. TIL therapy involves surgically extricating a tumor mass, separating the TILs from the tumor cells on a density gradient, expanding the lymphocytes in immunostimulatory in vitro conditions and reinfusing the activated killer cells back into the patient [120, 121]. Mouse models contrasting the antitumor efficacy of TIL therapy to LAK therapy showed that TIL therapy had approximately a one hundred fold greater tumoricidal effect [122, 123]. A possible reason why TILs had an augmented tumor eradicating effect is that this therapy activates only lymphocytes that have recognized the tumor and are reacting to it. In the clinic, results using TIL have been fair, with reproducible responses in approximately 20% of melanoma patients [124]. A means of augmenting the efficacy of TILs is to enhance their killing potential by transfecting them with cDNA to TNF [125]. Thus in one embodiment of the disclosure, tumor endothelial targeting vaccines are utilized to overcome cancer endothelial mediated immune evasion of the tumor, which potentiates the ability of the vaccine-induced T cells to kill tumors.

Numerous means of stimulating immunity to tumor associated endothelial cells are known in the art. In one embodiment, growth factors, growth factor receptors, or antigens associated with tumor endothelial cells are chosen for production of a vaccine. Active immunization against tumor endothelium by vaccinating against proliferating endothelium or markers found on tumor endothelium has provided promising preclinical data. Specifically, in animal models it has been reported that immunization to antigens specifically found on tumor vasculature can lead to tumor regression. Studies have been reported using the following antigens: survivin [126-128], endosialin [129], and xenogeneic FGF2R [130], VEGF [131], VEGF-R2 [132], MMP-2 [133], and endoglin [134, 135]. Human trials have been conducted utilizing human umbilical vein endothelial (HUVEC) cells as tumor antigens, with responses being reported in patients. In one report describing a 17-patient trial, Tanaka et al demonstrated that HUVEC vaccine therapy significantly prolonged tumor doubling time and inhibited tumor growth in patients with recurrent glioblastoma, inducing both cellular and humoral responses against the tumor vasculature without any adverse events or noticeable toxicities [136].

For example, in one study description of optimization of endothelial cell based vaccines was described. The authors of the study utilized human umbilical vein endothelial cells (HUVEC), which were prepared in different ways. The following were specifically tested: 1) paraformaldehyde-fixed HUVEC; 2) glutaraldehyde-fixed HUVEC; 3) HUVEC lysate and; 4) live HUVEC; these four commonly used antigen forms were used to prepare vaccines named Para-Fixed-EC, Glu-Fixed-EC, Lysate-EC, and Live-EC, respectively. The investigators showed that Live-EC exhibited the most favorable anti-tumor growth and metastasis effects among the four vaccines in both H22 hepatocellular carcinoma and Lewis lung cancer models. High titer anti-HUVEC antibodies were detected in Live-EC immunized mice sera, and the immune sera of Live-EC group could significantly inhibit HUVEC proliferation and tube formation. Moreover, T cells isolated from Live-EC immunized mice exhibited strong cytotoxicity against HUVEC cells, with an increasing IFN-γ and decreasing Treg production in Live-EC immunized mice. Finally, CD31 immunohistochemical analysis of the excised tumors verified a significant reduction in vessel density after Live-EC vaccination, which was in accordance with the anti-tumor efficiency. Taken together, all the results proved that live HUVEC was the most effective antigen form to induce robust HUVEC specific antibody and CTL responses, which are known to lead to the significant inhibition of tumor growth and metastasis [137]. Accordingly, in one embodiment of the disclosure, live HUVEC cells are utilized as a vaccine for stimulation of immunity towards tumor endothelial cells, wherein in specific embodiments this stimulation of immunity results in sensitization of the tumor to conventional cancer vaccines that induce activation of T cells or B cells. Furthermore, in specific embodiments, means of overcoming the immune privileged state of the tumor endothelium by means of selectively inhibiting the tumor endothelial immune suppressive state are encompassed herein. Elimination of immune suppressive state can be accomplished by induction of killing of tumor endothelium but can also be accomplished by blocking of suppressive factors, proteins, and/or peptides found on the tumor endothelium. For example, in one embodiment the vaccination with tumor endothelium targeting immunogens can lead to antibodies to molecules such as FasL, which block the ability of the FasL on the tumor endothelium to induce killing of T cells attempting to infiltrate the tumor. Means of inactivation of immune suppressive molecules found on tumor endothelium include antibody blockade of function, generation of coagulation on the surface of the tumor endothelium, as well as complement activation on the surface of the tumor endothelium.

In particular embodiments, addition of various adjuvants may be used to increase immunity of vaccines whose role is to stimulate immunity to tumor endothelium. Various adjuvants are known in the art, including various agonists of toll like receptors. Particular adjuvants include lipopolysaccharide an activator of TLR-4, Poly IC, a TLR-3 agonist, imiquimod a TLR-7 agonist, and CpG motifs such as TLR-9. Other adjuvants useful for the practice of at least some methods of the disclosure include Freund's Complete Adjuvant, Freund's Incomplete Adjuvant, BCG, and also loading on antigen presenting cells. In one embodiment, adjuvants are selected from the group consisting of Cationic liposome-DNA complex JVRS-100, aluminum hydroxide, aluminum phosphate vaccine, aluminum potassium sulfate adjuvant, Alhydrogel, ISCOM(s), Freund's Complete Adjuvant, Freund's Incomplete Adjuvant, CpG DNA Vaccine Adjuvant, Cholera toxin, Cholera toxin B subunit liposomes, Saponin, DDA, Squalene-based Adjuvants, Etx B subunit, IL-12, LTK63 Vaccine Mutant Adjuvant, TiterMax Gold Adjuvant, Ribi Vaccine Adjuvant, Montanide ISA 720 Adjuvant, Corynebacterium-derived P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, Adjumerm, Algal Glucan, Bay R1005, Theramide®, thalidomide, Stearyl Tyrosine, Specol, Algammulin, Avridine®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin-1β, Interleukin-2, Interleukin-7, Sclavo peptide, Rehydragel LV, Rehydragel HPA, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(I:C), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteoliposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-E112K, Matrix-S, and a combination thereof. One type of antigen presenting cell useful as an adjuvant for the practice of methods of the disclosure are dendritic cells. Numerous means of generating DC are described in the art. In one embodiment, peripheral blood mononuclear cells (PBMC) are extracted and subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7.

In some embodiments, augmentation of endogenous cellular vaccines is performed by stimulating immunity to tumor endothelium. The immunity towards said tumor endothelium is aimed to allow a sensitization of the tumor to T cells. In other embodiments, targeting of the tumor endothelium is performed to overcome the ability of the endothelium to protect the tumor.

In one embodiment of the disclosure, endogenous release of tumor antigens is used as a source of tumor antigens. One method of inducing localized tumor cell death is transarterial chemoembolization (TACE), or otherwise defined as transcatheter chemoembolization, which is a clinical procedure used primarily for treating primary and secondary liver cancer [138]. TACE is usually employed when standard therapy has failed or is known to be ineffective. TACE combines the advantages of intra-arterial chemotherapy with the fact that embolization of the portal artery induces a preferential “starvation” of the tumor while sparing non-malignant hepatic tissue. Specifically, it is established that intra-arterial delivery of chemotherapy to the liver results in a tenfold higher intra-tumoral concentration as compared to administration through the portal vein [139]. This is due in part to the observation that both primary and secondary liver tumors derive their blood supply preferentially from the hepatic artery [140]. Anecdotal evidence suggested that embolization caused by thrombosis of the catheter during delivery of intraarterial chemotherapy as beneficial for inducing an improved tumor response. This prompted investigators to use surgical ablation [141] or angiographic embolization [142-144] to induce localized necrosis. Unfortunately, this approach in absence of chemotherapy caused little effect on long-term survival. Therefore the advantage of TACE is that both localized delivery of chemotherapy to the tumor occurs, while at the same time the tumor blood flow is embolized, causing local tumor necrosis [145]. In some embodiments, these advantages are used for the practice of methods of the disclosure in which combination of TACE with cancer endothelial cell vaccination is performed.

The stimulation of cancer immunity is a result, in one embodiment, by release of tumor cell antigens from dying cancer cells. Globally speaking, apoptotic cell death is associated with anti-inflammatory and in some cases tolerogenesis, whereas necrotic cell death is perceived by the immune system as a “danger signal”, and is associated with immune activation [146-150]. Specific examples of the anti-inflammatory aspects of apoptotic cell death include the following: the production of IL-10 by apoptotic monocytes [151]; suppression of inflammatory cytokines by apoptotic bodies in vitro [152, 153]; observations that administration of apoptotic but not necrotic cell bodies can actually endow macrophages with active immune suppressive properties [154]; and clinically administered apoptotic blood cells have been demonstrated successful for treatment of inflammation associated with advanced heart failure in a recent Phase II trial [155]. Conversely, cellular necrosis is associated with release of a variety of innate immune activation signals such as heat shock proteins [156-158], HMGB1 [159], mRNA with endogenous secondary structures [160], and even DNA complexed with endogenous factors such as natural antibodies [161, 162]. Therefore the induction of cellular necrosis caused by TACE induces a release of tumor antigens, which is picked up by the immune system. The release of tumor antigens in such situations is reported in the literature [163], however taking advantage of this antigen release in the therapeutic context has not been accomplished to date. Although in the case of hepatocellular carcinoma, the tumor itself [164-167] and host cells infiltrating the tumor are known to be immune suppressive [168], the microenvironment in which TACE induces cellular necrosis is also normally immune suppressive. It is known that intrahepatic administration of antigens results in systemic immune deviation towards weak cellular immunity [169]. For example, it was demonstrated that administration of donor cells into the hepatic circulation resulted in prolonged, donor specific, graft acceptance in various models of transplantation [170-174]. The localized immune suppressive effects of the liver are known to the transplant clinician in that liver transplant recipients require a lower degree of immune suppression as compared to other organs. Additionally, in various rodent strain combinations hepatic grafts are spontaneously accepted, while cardiac or renal are rejected [175-177]. At a cellular level this is explained by the presence of immature hepatic DC [178, 179], the tolerogenic potential of liver sinusoidal endothelial cells [180, 181], as well as natural killer T cells with a predisposition for releasing IL-4 [182, 183]. Based on this, a release of tumor antigens within the hepatic microenvironment is postulated to cause a Th2, or immune regulatory shift, thereby not only failing to initiate protective immunity towards micrometastasis, but in some cases maybe even increasing the rate of tumor growth, through the phenomena of “tumor enhancement” described by Prehn [184]. Accordingly, it is one object of the present disclosure to stimulate Th1 immunity, which is cell-based, and avoid antibody based immunity to the tumor cells.

One specific embodiment of the disclosure involves modification of the TACE procedure in order to induce a systemic anti-tumor immunological effect. Specifically, patients are selected to meet the criteria for TACE, such as including the following: a) Adequate hepatic function; b) Patient portal vein circulation (confirmed during the venous phase of celiac or superior mesenteric angiogram); and c) Adequate renal function. Generally, only patients without cirrhosis or in Child group A or B disease are considered, however depending on experience of the practicing physician other groups may be included in the procedure as discussed by Shah et al [185]. The TACE procedure may be performed either using a selective or superselective means. Patients selected to undergo the procedure receive 10 mg of phytonadione intravenously prior to the procedure (the intravenous injection should be administered slowly). Femoral catheterization and positioning of the catheter is performed. Premedication is with Lorazepam (Wyeth Laboratories, UK) 0.25 mg/kg orally 1 hour before the procedure to counter anxiety. An intra-arterial injection of 30-40 mg of 1% lidocaine is used for analgesia.

The following ingredients are made into an emulsion by repeatedly emptying and filling a syringe over 10 minutes: 10 mL of Lipiodol Ultrafluid (Mallinckrodt Medical, UK), 5 mL Omnipaque 300 (Amersham Health, UK; water-soluble contrast aids in emulsifying the mixture), 50 mg doxorubicin and clinical grade Poly (IC) stabilized with carboxymethylcellulose at a concentration between 0.025 mg/m2 to 12 mg/m2, preferably at a concentration of 0.2 mg/m2. Intraarterial injection is administered under direct visualization to prevent reflux into gastroduodenal or splenic vessels. Embolization is performed with Ultra Ivalon 250-400 μm (Laboratories Nycomed SA). Intravenous cefuroxime (750 mg) and metronidazole (500 mg) are administered 3 times per day for 5 days. These antibiotics are given as prophylaxis against septicemia and liver abscess formation. Subsequent to administration patients are admitted to a high-dependency ward and should be mobilized after 6 hours of bedrest. Postoperative analgesia is administered if and when required by the patient. Patients also receive ranitidine (an H2 antagonist) intravenously 3 times per day until they begin eating. Patients are discharged home after 5 days or when their systemic symptoms begin resolving. In order to monitor success of the procedure nonenhanced and enhanced CT examinations are performed 10-14 days following embolization. Furthermore, alpha-fetoprotein levels are evaluated at the 6-week outpatient review. If the TACE procedure is successful (>50% lipiodol uptake in necrotic tumor demonstrated on the postprocedural CT scan), the embolization is repeated in 6-8 weeks. Immunological monitoring is performed by assessing levels of interferon alpha production using ELISA during the 12, 24, and 72 hour time periods. Additionally, DTH, cellular and antibody responses may be measured using pre-defined antigens representative of the tumor type.

A variety of chemotherapeutic agents may be used in some embodiments of the disclosure. Specifically, chemotherapeutic agents that induce upregulation of costimulatory molecules may be utilized. One example of such an agent is melphalan, which induces expression of CD80 on both tumor cells [186], as well as non-tumor B cells [187]. In addition, a wide variety of chemotherapeutic agents are known in the art. These include the following: alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; and/or capecitabine.

A TACE-modification procedure may be utilized as one embodiment. Additionally modifications may be made to increase efficacy of anti-tumor response being mediated. Particularly, a wide variety of agents can be administered to an individual prior to the TACE procedure in order to increase general immunological status, and specifically, T cell, NK cell, and NKT cell functions. One particular modification may involve the administration of an anti-oxidant capable of reversing immune suppression seen in many cancer patients. Immune suppression by cancer has been well-documented in advanced cancer patients possessing a variety of malignancies [188-195]. Correlation between immune suppression and poor prognosis has been extensively noted [196-198]. Several means of tumor suppression of immune response are known. For example, a variety of tumor cells possess the ability to induce cleavage of the T cell receptor zeta (TCR-ζ) chain through a caspase-3 dependent manner [199, 200]. Because TCR-ζ is critical for signal transduction, host T cells become unable to respond to tumor antigens. Originally, the TCR-ζ cleavage was described in tumor bearing mice [201, 202] and subsequently in patients [203-208]. The correlation between suppressed TCR-ζ and suppressed IFN-g production has been reported, implying functional consequences [204]. The cause of TCR-ζ suppression has been attributed, at least in part, to reactive oxygen radicals produced by: A) The inflammatory activity occurring inside the tumor (it is well established that there is a constant area of necrosis intratumorally); B) Macrophages associated with the tumor; and C) Neutrophils activated directly by the tumor, or by the tumor associated macrophages.

Tumors are usually associated with macrophage infiltration, and this is correlated with tumor stage and is believed to contribute to tumor progression by stimulation of angiogenesis [209-211]. Cytokines such as M-CSF [209] and VEGF [212] produced by tumor infiltrating macrophages are essential for tumor progression to malignancy. In fact, tumors implanted into M-CSF deficient op/op mice (that lack macrophages) do not metastasize or become vascularized [213]. Tumor-associated macrophages possess an activated phenotype and release various inflammatory mediators such as cyclo-oxygenase metabolites [214, 215], TNF [216], and IL-6 [217] which lead to increased levels of oxidative stress produced by host immune cells. In addition, tumor associated macrophages themselves produce large amounts of free radicals such as NO, OH, and H2O2 [218-220]. The high levels of macrophage activation in cancer patients is illustrated by high serum levels of neopterin, a tryptophan metabolite that is associated with poor prognosis [221]. In addition to oxidative stress elaborated by tumor associated macrophages, the presence of the tumor itself causes systemic changes associated with chronic inflammation. Erythrocyte sedimentation ration, C-reactive protein and IL-6 are markers of inflammatory stress used to designate progression of pathological immune diseases such as arthritis [222, 223]. Interestingly advanced cancer patients possess all of these inflammatory markers [224-228]. Another marker of chronic inflammation is decreased albumin synthesis by the liver, this is also seen in cancer patients and is believed to contribute, at least in part, to cachexia [229, 230]. In addition, the inflammatory marker fibrinogen D-dimers is also higher in cancer patients as opposed to controls [231-233]. Schmielau et al reported that in patients with a variety of cancers, activated neutrophils are circulating in large numbers [203]. These neutrophils secrete reactive oxygen radicals such as hydrogen peroxide, which trigger suppression of TCR-ζ and IFN-g production. This was demonstrated by co-incubation of the neutrophils from cancer patients with lymphocytes from healthy volunteer. A profound suppression of TCR-ζ expression was seen. Evidence for the critical role of hydrogen peroxide was shown by the fact that addition of catalase suppressed TCR-ζ downregulation. A simple method of assessing the number of circulating activated neutrophils was described therein in which there is collection of peripheral blood from patients, spinning of the blood on a density gradient such as Ficoll, and collection of the lymphocyte fraction. While in healthy volunteers the lymphocyte fraction contained primarily lymphocytes, in cancer patients the lymphocyte fraction contained both lymphocytes and a large number of neutrophils. The reason why these neutrophils are present in the lymphocyte fraction is because activation alters their density so that they co-purify differently on the gradient. One indication of the importance of activated neutrophils to cancer progression is provided by Tabuchi et al who show that removal of granulocytes from the peripheral blood of cancer patients resulted in reduced tumor size, unfortunately, the study was performed in only 2 patients [234]. As a mechanism to compensate for immune over-activation, mediators of inflammation have immune suppressive properties. This is best illustrated in the immune suppression seen following immune hyperactivation such as in septic shock. Following the primary scepticemia, patients are systemically immune compromised due to circulating immune suppressive factors that are released in response to the inflammatory stress. This suppression is termed compensatory anti-inflammatory response syndrome (CARS) and is associated with many opportunistic infections and deactivation [235]. The clinical importance of CARS immune suppression is seen in that sepsis survivors show normal T-cell proliferation and IL-2 release, whereas those that succumb possess suppressed T cell responses [236]. Interestingly immune suppressive mediators associated with CARS such as PGE2, TGF-b, and IL-10 are also associated with cancer-induced immune suppression [237]. The role of oxidative stress in sepsis-induced immune suppression was recently demonstrated in experiments where administration of antioxidants (ascorbic acid or n-acetylcysteine) to animals undergoing experimental sepsis blocked immune suppression [238]. Another example of the potential for antioxidants to stimulate immune response in an inflammatory condition is in patients with Duke's C and D colorectal cancer who were administered of a daily dose of 750 mg of vitamin E for 2 weeks. This resulted in restoration of IFN-g and IL-2 production [239]. The problem of uncontrolled inflammation is seen in sepsis. Although as a monotherapy n-acetylcysteine has little clinical effect, therapeutic administration of n-acetylcysteine results in suppression of the constitutively activated neutrophils seen in these patients [240]. Administration of n-acetylcysteine to smokers results in suppression of markers of oxidative stress [241]. Furthermore, oral n-acetylcysteine administration blocks angiogenesis and suppresses growth of Kaposi Sarcoma [242]. Accordingly, a method of preparing the host for the TACE procedure includes administration of n-acetylcysteine at a concentration sufficient to decrease the tumor associated suppression of T cell activity. In specific embodiments, such a concentration ranges between 1-10 grams per day, such as 4-6 grams administered intravenously for a period of type sufficient to normalize production of IFN-g from PBMC of cancer patients upon ex vivo stimulation. One skilled in the art will understand that n-acetylcysteine is just one example of a compound suitable for reversion of oxidative-stress associated immune suppression. Numerous other compounds may be used, for example ascorbic acid [243-245], co-enzyme Q10 in combination with vitamin E and alpha-lipoic acid [246], genistein [247] or resveratrol [248].

In some embodiments, dendritic cells are utilized to induce an augmented immune response subsequent to TACE induced release of antigens. In other embodiments, dendritic cells are administered close to the proximity of the TACE induced cell death. In one embodiment, DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor endothelial lysate or with fibroblast derived endothelial cells. Pulsing may be performed by incubation of lysates with dendritic cells, or may be generated by fusion of immature dendritic cells with fibroblast derived endothelial cells. Means of generating hybridomas or cellular fusion products are known in the art and include electrical pulse mediated fusion, or stimulation of cellular fusion by treatment with polyethelene glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of fibroblast-derived endothelial cell pulsed dendritic cells is utilized as a polyvalent vaccine, whereas subsequent to administration antibody or T cell responses are assessed for induction of antigen specificity, and peptides corresponding to immune response stimulated are used for further immunization to focus the immune response. Protocols useful for generation of dendritic cells have been previously used to generate immunity to a variety of tumors and are disclosed in the following which are incorporated by reference in melanoma [249-300], soft tissue sarcoma [301], thyroid [302-304], glioma [305-326], multiple myeloma, [327-335], lymphoma [336-338], leukemia [339-346], as well as liver [347-352], lung [353-366], ovarian [367-370], and pancreatic cancer [371-373].

### REFERENCES

The following references, to the extent that they provide exemplary procedural or other details supplementary to those set forth herein, are specifically incorporated herein by reference.
1. Ryschich, E., et al., Control of T-cell-mediated immune response by HLA class I in human pancreatic carcinoma. Clin Cancer Res, 2005. 11(2 Pt 1): p. 498-504.
2. Raspollini, M. R., et al., Tumour-infiltrating gamma/delta T-lymphocytes are correlated with a brief disease-free interval in advanced ovarian serous carcinoma. Ann Oncol, 2005. 16(4): p. 590-6.
3. Chiba, T., et al., Intraepithelial CD8+ T-cell-count becomes a prognostic factor after a longer follow-up period in human colorectal carcinoma: possible association with suppression of micrometastasis. Br J Cancer, 2004. 91(9): p. 1711-7.
4. Astigiano, S., et al., Eosinophil granulocytes account for indoleamine 2,3-dioxygenase-mediated immune escape in human non-small cell lung cancer. Neoplasia, 2005. 7(4): p. 390-6.
5. Whiteside, T. L., Down-regulation of zeta-chain expression in T cells: a biomarker of prognosis in cancer? Cancer Immunol Immunother, 2004. 53(10): p. 865-78.
6. Rosenberg, S. A. and M. E. Dudley, Cancer regression inpatients with metastatic melanoma after the transfer of autologous antitumor lymphocytes. Proc Natl Acad Sci USA, 2004. 101 Suppl 2: p. 14639-45.
7. Ichim, T. E., et al., Induction of tumor inhibitory anti-angiogenic response through immunization with interferon Gamma primed placental endothelial cells: ValloVax. J Transl Med, 2015. 13: p. 90.
8. Wagner, S. C., et al., Cancer anti-angiogenesis vaccines: Is the tumor vasculature antigenically unique? J Transl Med, 2015. 13: p. 340.
9. Wagner, S. C., et al., Safety of targeting tumor endothelial cell antigens. J Transl Med, 2016. 14: p. 90.
10. Wagner, S. C., et al., Induction and characterization of anti-tumor endothelium immunity elicited by ValloVax therapeutic cancer vaccine. Oncotarget, 2017. 8(17): p. 28595-28613.
11. Takahashi, S., et al., Fine-Tuning Approach for Segmentation of Gliomas in Brain Magnetic Resonance Images with a Machine Learning Method to Normalize Image Differences among Facilities. Cancers (Basel), 2021. 13(6).
12. Zhu, X. Y., et al., Mesenchymal stem cells and endothelial progenitor cells decrease renal injury in experimental swine renal artery stenosis through different mechanisms. Stem Cells, 2013. 31(1): p. 117-25.
13. Lopez-Holgado, N., et al., Short-term endothelial progenitor cell colonies are composed of monocytes and do not acquire endothelial markers. Cytotherapy, 2007. 9(1): p. 14-22.
14. Achyut, B. R. and A. S. Arbab, Myeloid Derived Suppressor Cells: Fuel the Fire. Biochem Physiol, 2014. 3(3): p. e123.
15. Sander, A. L., et al., Systemic transplantation of progenitor cells accelerates wound epithelialization and neovascularization in the hairless mouse ear wound model. J Surg Res, 2011. 165(1): p. 165-70.
16. Fortini, C., et al., Circulating stem cell vary with NYHA stage in heart failure patients. J Cell Mol Med, 2011. 15(8): p. 1726-36.
17. Tardif, K., et al., A phosphorylcholine-modified chitosan polymer as an endothelial progenitor cell supporting matrix. Biomaterials, 2011. 32(22): p. 5046-55.
18. Campioni, D., et al., In vitro characterization of circulating endothelial progenitor cells isolated from patients with acute coronary syndrome. PLoS One, 2013. 8(2): p. e56377.
19. Nowak, W. N., et al., Number of circulating pro-angiogenic cells, growth factor and anti-oxidative gene profiles might be altered in type 2 diabetes with and without diabetic foot syndrome. J Diabetes Investig, 2014. 5(1): p. 99-107.
20. Obtulowicz, P., et al., Induction of Endothelial Phenotype From Wharton's Jelly-Derived MSCs and Comparison of Their Vasoprotective and Neuroprotective Potential With Primary WJ-MSCs in CA1 Hippocampal Region Ex Vivo. Cell Transplant, 2016. 25(4): p. 715-27.
21. Rohde, E., et al., Blood monocytes mimic endothelial progenitor cells. Stem Cells, 2006. 24(2): p. 357-67.
22. Bagley, R. G., et al., Pericytes and endothelial precursor cells: cellular interactions and contributions to malignancy. Cancer Res, 2005. 65(21): p. 9741-50.
23. Finney, M. R., et al., Direct comparison of umbilical cord blood versus bone marrow-derived endothelial precursor cells in mediating neovascularization in response to vascular ischemia. Biol Blood Marrow Transplant, 2006. 12(5): p. 585-93.
24. Neumuller, J., et al., Immunological and ultrastructural characterization of endothelial cell cultures differentiated from human cord blood derived endothelial progenitor cells. Histochem Cell Biol, 2006. 126(6): p. 649-64.
25. Nguyen, V. A., et al., Endothelial cells from cord blood CD133+CD34+ progenitors share phenotypic, functional and gene expression profile similarities with lymphatics. J Cell Mol Med, 2009. 13(3): p. 522-34.
26. Kamei, N., et al., Lnk deletion reinforces the function of bone marrow progenitors in promoting neovascularization and astrogliosis following spinal cord injury. Stem Cells, 2010. 28(2): p. 365-75.
27. Kuliczkowski, W., et al., Endothelial progenitor cells and left ventricle function inpatients with acute myocardial infarction: potential therapeutic considerations. Am J Ther, 2012. 19(1): p. 44-50.
28. Damaghi, M. and R. Gillies, Phenotypic changes of acid adapted cancer cells push them toward aggressiveness in their evolution in the tumor microenvironment. Cell Cycle, 2016: p. 0.
29. Lobo, R. C., et al., Glucose Uptake and Intracellular pH in a Mouse Model of Ductal Carcinoma In situ (DCIS) Suggests Metabolic Heterogeneity. Front Cell Dev Biol, 2016. 4: p. 93.
30. An, S., et al., Amino Acid Metabolism Abnormity and Microenvironment Variation Mediated Targeting and Controlled Glioma Chemotherapy. Small, 2016.
31. Avnet, S., et al., Altered pH gradient at the plasma membrane of osteosarcoma cells is a key mechanism of drug resistance. Oncotarget, 2016.
32. Huang, S., et al., Acidic extracellular pH promotes prostate cancer bone metastasis by enhancing PC-3 stem cell characteristics, cell invasiveness and VEGF-induced vasculogenesis of BM-EPCs. Oncol Rep, 2016. 36(4): p. 2025-32.
33. Carnero, A. and M. Lleonart, The hypoxic microenvironment: A determinant of cancer stem cell evolution. Bioessays, 2016. 38 Suppl 1: p. S65-74.
34. Pellegrini, P., et al., Tumor acidosis enhances cytotoxic effects and autophagy inhibition by salinomycin on cancer cell lines and cancer stem cells. Oncotarget, 2016. 7(24): p. 35703-35723.
35. Bohme, I. and A. K. Bosserhoff, Acidic tumor microenvironment in human melanoma. Pigment Cell Melanoma Res, 2016. 29(5): p. 508-23.
36. Gentric, G., V. Mieulet, and F. Mechta-Grigoriou, Heterogeneity in Cancer Metabolism: New Concepts in an Old Field. Antioxid Redox Signal, 2016.
37. Ge, Y., et al., Preferential extension of short telomeres induced by low extracellular pH. Nucleic Acids Res, 2016. 44(17): p. 8086-96.
38. Quail, D. F., et al., The tumor microenvironment underlies acquired resistance to CSF-JR inhibition in gliomas. Science, 2016. 352(6288): p. aad3018.
39. Li, X., et al., The altered glucose metabolism in tumor and a tumor acidic microenvironment associated with extracellular matrix metalloproteinase inducer and monocarboxylate transporters. Oncotarget, 2016. 7(17): p. 23141-55.
40. Zhao, C., et al., Tumor Acidity-Induced Sheddable Polyethylenimine-Poly(trimethylene carbonate)/DNA/Polyethylene Glycol-2,3-Dimethylmaleicanhydride Ternary Complex for Efficient and Safe Gene Delivery. ACS Appl Mater Interfaces, 2016. 8(10): p. 6400-10.
41. Riemann, A., et al., Acidosis Promotes Metastasis Formation by Enhancing Tumor Cell Motility. Adv Exp Med Biol, 2016. 876: p. 215-20.
42. Ravi shankar, B., et al., The amino acid sensor GCN2 inhibits inflammatory responses to apoptotic cells promoting tolerance and suppressing systemic autoimmunity. Proc Natl Acad Sci USA, 2015. 112(34): p. 10774-9.
43. Walls, J., L. Sinclair, and D. Finlay, Nutrient sensing, signal transduction and immune responses. Semin Immunol, 2016.
44. Munn, D. H., et al., GCN2 kinase in T cells mediates proliferative arrest and anergy induction in response to indoleamine 2,3-dioxygenase. Immunity, 2005. 22(5): p. 633-42.
45. Ishimura, R., et al., Activation of GCN2 kinase by ribosome stalling links translation elongation with translation initiation. Elife, 2016. 5.
46. Lageix, S., et al., Enhanced interaction between pseudokinase and kinase domains in Gcn2 stimulates eIF2alpha phosphorylation in starved cells. PLoS Genet, 2014. 10(5): p. e1004326.
47. Metz, R., et al., IDO inhibits a tryptophan sufficiency signal that stimulates mTOR: A novel IDO effector pathway targeted by D-1-methyl-tryptophan. Oncoimmunology, 2012. 1(9): p. 1460-1468.
48. Manlapat, A. K., et al., Cell-autonomous control of interferon type I expression by indoleamine 2,3-dioxygenase in regulatory CD19+ dendritic cells. Eur J Immunol, 2007. 37(4): p. 1064-71.
49. Sharma, M. D., et al., Plasmacytoid dendritic cells from mouse tumor-draining lymph nodes directly activate mature Tregs via indoleamine 2,3-dioxygenase. J Clin Invest, 2007. 117(9): p. 2570-82.
50. Forouzandeh, F., et al., Differential immunosuppressive effect of indoleamine 2,3-dioxygenase (IDO) on primary human CD4+ and CD8+ T cells. Mol Cell Biochem, 2008. 309(1-2): p. 1-7.
51. Wek, R. C., H. Y. Jiang, and T. G. Anthony, Coping with stress: eIF2 kinases and translational control. Biochem Soc Trans, 2006. 34(Pt 1): p. 7-11.
52. Xia, X. J., et al., Autophagy mediated by arginine depletion activation of the nutrient sensor GCN2 contributes to interferon-gamma-induced malignant transformation of primary bovine mammary epithelial cells. Cell Death Discov, 2016. 2: p. 15065.
53. Xia, X., et al., Arginine Supplementation Recovered the IFN-gamma-Mediated Decrease in Milk Protein and Fat Synthesis by Inhibiting the GCN2/eIF2alpha Pathway, Which Induces Autophagy in Primary Bovine Mammary Epithelial Cells. Mol Cells, 2016. 39(5): p. 410-7.
54. Vynnytska-Myronovska, B. O., et al., Arginine starvation in colorectal carcinoma cells: Sensing, impact on translation control and cell cycle distribution. Exp Cell Res, 2016. 341(1): p. 67-74.
55. Marion, V., et al., Arginine deficiency causes runting in the suckling period by selectively activating the stress kinase GCN2. J Biol Chem, 2011. 286(11): p. 8866-74.
56. Rodriguez, P. C., D. G. Quiceno, and A. C. Ochoa, L-arginine availability regulates T-lymphocyte cell-cycle progression. Blood, 2007. 109(4): p. 1568-73.
57. Bunpo, P., et al., The eIF2 kinase GCN2 is essential for the murine immune system to adapt to amino acid deprivation by asparaginase. J Nutr, 2010. 140(11): p. 2020-7.
58. Bunpo, P., et al., GCN2 protein kinase is required to activate amino acid deprivation responses in mice treated with the anti-cancer agent L-asparaginase. J Biol Chem, 2009. 284(47): p. 32742-9.
59. Balasubramanian, M. N., E. A. Butterworth, and M. S. Kilberg, Asparagine synthetase: regulation by cell stress and involvement in tumor biology. Am J Physiol Endocrinol Metab, 2013. 304(8): p. E789-99.
60. Wilson, G. J., et al., The eukaryotic initiation factor 2 kinase GCN2 protects against hepatotoxicity during asparaginase treatment. Am J Physiol Endocrinol Metab, 2013. 305(9): p. E1124-33.
61. Ye, J., et al., GCN2 sustains mTORC1 suppression upon amino acid deprivation by inducing Sestrin2. Genes Dev, 2015. 29(22): p. 2331-6.
62. Reinert, R. B., et al., Role of glutamine depletion in directing tissue-specific nutrient stress responses to L-asparaginase. J Biol Chem, 2006. 281(42): p. 31222-33.
63. Drogat, B., et al., Acute L-glutamine deprivation compromises VEGF-a upregulation in A549/8 human carcinoma cells. J Cell Physiol, 2007. 212(2): p. 463-72.
64. Eil, R., et al., Ionic immune suppression within the tumour microenvironment limits T cell effector function. Nature, 2016. 537(7621): p. 539-543.
65. Young, A., et al., Co-inhibition of CD73 and A2AR Adenosine Signaling Improves Anti-tumor Immune Responses. Cancer Cell, 2016. 30(3): p. 391-403.
66. Hay, C. M., et al., Targeting CD73 in the tumor microenvironment with MEDI9447. Oncoimmunology, 2016. 5(8): p. e1208875.
67. Montalban Del Barrio, I., et al., Adenosine-generating ovarian cancer cells attract myeloid cells which differentiate into adenosine-generating tumor associated macrophages—a self-amplifying, CD39-and CD73-dependent mechanism for tumor immune escape. J Immunother Cancer, 2016. 4: p. 49.
68. Gaudreau, P. O., et al., CD73-adenosine reduces immune responses and survival in ovarian cancer patients. Oncoimmunology, 2016. 5(5): p. e1127496.
69. Takenaka, M. C., S. Robson, and F. J. Quintana, Regulation of the T Cell Response by CD39. Trends Immunol, 2016. 37(7): p. 427-39.
70. Ohta, A., A Metabolic Immune Checkpoint: Adenosine in Tumor Microenvironment. Front Immunol, 2016. 7: p. 109.
71. Mandapathil, M., Adenosine-mediated immunosuppression inpatients with squamous cell carcinoma of the head and neck. HNO, 2016. 64(5): p. 303-9.
72. Vaupel, P. and G. Multhoff, Adenosine can thwart antitumor immune responses elicited by radiotherapy: Therapeutic strategies alleviating protumor ADO activities. Strahlenther Onkol, 2016. 192(5): p. 279-87.
73. Allard, D., et al., CD73-adenosine: a next-generation target in immuno-oncology. Immunotherapy, 2016. 8(2): p. 145-63.
74. Young, A., et al., Co-blockade of immune checkpoints and adenosine A2A receptor suppresses metastasis. Oncoimmunology, 2014. 3(10): p. e958952.
75. Zhang, B., et al., High expression of CD39/ENTPD1 in malignant epithelial cells of human rectal adenocarcinoma. Tumour Biol, 2015. 36(12): p. 9411-9.
76. Mandapathil, M., et al., Isolation of functional human regulatory T cells (Treg) from the peripheral blood based on the CD39 expression. J Immunol Methods, 2009. 346(1-2): p. 55-63.
77. Fishman, P., et al., Adenosine receptors and cancer. Handb Exp Pharmacol, 2009(193): p. 399-441.
78. Sun, X., et al., CD39/ENTPD1 expression by CD4+Foxp3+ regulatory T cells promotes hepatic metastatic tumor growth in mice. Gastroenterology, 2010. 139(3): p. 1030-40.
79. Turcotte, M., et al., CD73 is associated with poor prognosis in high-grade serous ovarian cancer. Cancer Res, 2015. 75(21): p. 4494-503.
80. Beavis, P. A., et al., Adenosine Receptor 2A Blockade Increases the Efficacy of Anti-PD-1 through Enhanced Antitumor T-cell Responses. Cancer Immunol Res, 2015. 3(5): p. 506-17.
81. Suda, T., et al., Molecular cloning and expression of the Fas ligand, a novel member of the tumor necrosis factor family. Cell, 1993. 75(6): p. 1169-78.
82. Suda, T. and S. Nagata, Purification and characterization of the Fas-ligand that induces apoptosis. J Exp Med, 1994. 179(3): p. 873-9.
83. Ichinose, M., et al., Fas ligand expression and depletion of T-cell infiltration in astrocytic tumors. Brain Tumor Pathol, 2001. 18(1): p. 37-42.
84. Ferguson, T. A. and T. S. Griffith, The role of Fas ligand and TNF-related apoptosis-inducing ligand (TRAIL) in the ocular immune response. Chem Immunol Allergy, 2007. 92: p. 140-54.
85. McKechnie, N. M., et al., Fas-ligand is stored in secretory lysosomes of ocular barrier epithelia and released with microvesicles. Exp Eye Res, 2006. 83(2): p. 304-14.
86. Sano, Y. and C. Sotozono, Role of Fas ligand in ocular tissue. Cornea, 2002. 21(2 Suppl 1): p. S30-2.
87. Taylor, A. W., Ocular immunosuppressive microenvironment. Chem Immunol, 1999. 73: p. 72-89.
88. Brignole, F., et al., Expression of Fas-Fas ligand antigens and apoptotic marker APO2.7 by the human conjunctival epithelium. Positive correlation with class II HLA DR expression in inflammatory ocular surface disorders. Exp Eye Res, 1998. 67(6): p. 687-97.
89. Griffith, T. S., et al., Fas ligand-induced apoptosis as a mechanism of immune privilege. Science, 1995. 270(5239): p. 1189-92.
90. Inui, Y., et al., Fas-ligand expression on nucleus pulposus begins in developing embryo. Spine (Phila Pa. 1976), 2004. 29(21): p. 2365-9.
91. Takada, T., et al., Fas ligand exists on intervertebral disc cells: a potential molecular mechanism for immune privilege of the disc. Spine (Phila Pa. 1976), 2002. 27(14): p. 1526-30.
92. Sun, Z., et al., Fluoride reduced the immune privileged function of mouse Sertoli cells via the regulation of Fas/FasL system. Chemosphere, 2017. 168: p. 318-325.
93. Bayram, S., G. Kizilay, and Y. Topcu-Tarladacalisir, Evaluation of the Fas/FasL signaling pathway in diabetic rat testis. Biotech Histochem, 2016. 91(3): p. 204-11.
94. Ma, C., et al., Characterization of swine testicular cell line as immature porcine Sertoli cell line. In Vitro Cell Dev Biol Anim, 2016. 52(4): p. 427-33.
95. Assis, P. V., et al., Expression of FAS ligand in the ipsilateral and contralateral testicles of rats subjected to the torsion of the unilateral testicular cord. Acta Cir Bras, 2013. 28(7): p. 518-22.
96. Jana, K., et al., Ethanol induces mouse spermatogenic cell apoptosis in vivo through over-expression of Fas/Fas-L, p53, and caspase-3 along with cytochrome c translocation and glutathione depletion. Mol Reprod Dev, 2010. 77(9): p. 820-33.
97. Dufour, J. M., et al., Sertoli cell line lacks the immunoprotective properties associated with primary Sertoli cells. Cell Transplant, 2008. 17(5): p. 525-34.
98. Catalano, S., et al., Fas ligand expression in TM4 Sertoli cells is enhanced by estradiol “in situ” production. J Cell Physiol, 2007. 211(2): p. 448-56.
99. D'Abrizio, P., et al., Ontogenesis and cell specific localization of Fas ligand expression in the rat testis. Int J Androl, 2004. 27(5): p. 304-10.
100. Bart, J., et al., An oncological view on the blood-testis barrier. Lancet Oncol, 2002. 3(6): p. 357-63.
101. Lan, P., et al., Immune privilege induced by cotransplantation of islet and allogeneic testicular cells. Chin Med J (Engl), 2001. 114(10): p. 1026-9.
102. Filippini, A., et al., Control and impairment of immune privilege in the testis and in semen. Hum Reprod Update, 2001. 7(5): p. 444-9.
103. Xu, J. P., et al., Expression of Fas-Fas ligand in murine testis. Am J Reprod Immunol, 1999. 42(6): p. 381-8.
104. Guller, S. and L. LaChapelle, The role of placental Fas ligand in maintaining immune privilege at maternal-fetal interfaces. Semin Reprod Endocrinol, 1999. 17(1): p. 39-44.
105. Saporta, S., et al., Survival of rat and porcine Sertoli cell transplants in the rat striatum without cyclosporine-A immunosuppression. Exp Neurol, 1997. 146(2): p. 299-304.
106. Sanberg, P. R., et al., The testis-derived cultured Sertoli cell as a natural Fas-L secreting cell for immunosuppressive cellular therapy. Cell Transplant, 1997. 6(2): p. 191-3.
107. Choi, C. and E. N. Benveniste, Fas ligand/Fas system in the brain: regulator of immune and apoptotic responses. Brain Res Brain Res Rev, 2004. 44(1): p. 65-81.
108. Kubo, M., et al., Immunogenicity of human amniotic membrane in experimental xenotransplantation. Invest Ophthalmol Vis Sci, 2001. 42(7): p. 1539-46.
109. Kauma, S. W., et al., Placental Fas ligand expression is a mechanism for maternal immune tolerance to the fetus. J Clin Endocrinol Metab, 1999. 84(6): p. 2188-94.
110. McClure, R. F., C. J. Heppelmann, and C. V. Paya, Constitutive Fas ligandgene transcription in Sertoli cells is regulated by Spl. J Biol Chem, 1999. 274(12): p. 7756-62.
111. Uckan, D., et al., Trophoblasts express Fas ligand: a proposed mechanism for immune privilege in placenta and maternal invasion. Mol Hum Reprod, 1997. 3(8): p. 655-62.
112. Yu, J. S., et al., Intratumoral T cell subset ratios and Fas ligand expression on brain tumor endothelium. J Neurooncol, 2003. 64(1-2): p. 55-61.
113. Kawata, A., et al., Tumor-infiltrating lymphocytes and prognosis of hepatocellular carcinoma. Jpn J Clin Oncol, 1992. 22(4): p. 256-63.
114. Elder, D. E., et al., Neoplastic progression and prognosis in melanoma. Semin Cutan Med Surg, 1996. 15(4): p. 336-48.
115. Miwa, H., Identification and prognostic implications of tumor infiltrating lymphocytes—a review. Acta Med Okayama, 1984. 38(3): p. 215-8.
116. Lipponen, P. K., et al., Tumour infiltrating lymphocytes as an independent prognostic factor in transitional cell bladder cancer. Eur J Cancer, 1992. 29A(1): p. 69-75.
117. Ropponen, K. M., et al., Prognostic value of tumour-infiltrating lymphocytes (TILs) in colorectal cancer. J Pathol, 1997. 182(3): p. 318-24.
118. Ma, D. and M. J. Gu, Immune effect of tumor-infiltrating lymphocytes and its relation to the survival rate of patients with ovarian malignancies. J Tongii Med Univ, 1991. 11(4): p. 235-9.
119. Tomsova, M., et al., Prognostic significance of CD3+ tumor-infiltrating lymphocytes in ovarian carcinoma. Gynecol Oncol, 2008. 108(2): p. 415-20.
120. Filgueira, L., et al., Effects of different culture protocols on the expression of discrete T-cell receptor variable regions in human tumour infiltrating lymphocytes. Eur J Cancer, 1993. 29A(12): p. 1754-60.
121. Topalian, S. L., et al., Immunotherapy of patients with advanced cancer using tumor-infiltrating lymphocytes and recombinant interleukin-2: a pilot study. J Clin Oncol, 1988. 6(5): p. 839-53.
122. Spiess, P. J., J. C. Yang, and S. A. Rosenberg, In vivo antitumor activity of tumor-infiltrating lymphocytes expanded in recombinant interleukin-2. J Natl Cancer Inst, 1987. 79(5): p. 1067-75.
123. Yang, J. C., D. Perry-Lalley, and S. A. Rosenberg, An improved method for growing murine tumor-infiltrating lymphocytes with in vivo antitumor activity. J Biol Response Mod, 1990. 9(2): p. 149-59.
124. Whiteside, T. L., Cancer therapy with tumor-infiltrating lymphocytes: evaluation of potential and limitations. In Vivo, 1991. 5(6): p. 553-9.
125. Rosenberg, S. A., et al., The development of gene therapy for the treatment of cancer. Ann Surg, 1993. 218(4): p. 455-63; discussion 463-4.
126. Lladser, A., et al., Intradermal DNA electroporation induces survivin-specific CTLs, suppresses angiogenesis and confers protection against mouse melanoma. Cancer Immunol Immunother, 2010. 59(1): p. 81-92.
127. Xiang, R., et al., Oral DNA vaccines target the tumor vasculature and microenvironment and suppress tumor growth and metastasis. Immunol Rev, 2008. 222: p. 117-28.
128. Xiang, R., et al., A DNA vaccine targeting survivin combines apoptosis with suppression of angiogenesis in lung tumor eradication. Cancer Res, 2005. 65(2): p. 553-61.
129. Valdez, Y., M. Maia, and E. M. Conway, CD248: reviewing its role in health and disease. Curr Drug Targets, 2012. 13(3): p. 432-9.
130. Plum, S. M., et al., Generation of a specific immunological response to FGF-2 does not affect wound healing or reproduction. Immunopharmacol Immunotoxicol, 2004. 26(1): p. 29-41.
131. Wei, Y. Q., et al., Immunogene therapy of tumors with vaccine based on Xenopus homologous vascular endothelial growth factor as a model antigen. Proc Natl Acad Sci USA, 2001. 98(20): p. 11545-50.
132. Liu, J. Y., et al., Immunotherapy of tumors with vaccine based on quail homologous vascular endothelial growth factor receptor-2. Blood, 2003. 102(5): p. 1815-23.
133. Su, J. M., et al., Active immunogene therapy of cancer with vaccine on the basis of chicken homologous matrix metalloproteinase-2. Cancer Res, 2003. 63(3): p. 600-7.
134. Tan, G. H., et al., Active immunotherapy of tumors with a recombinant xenogeneic endoglin as a model antigen. Eur J Immunol, 2004. 34(7): p. 2012-21.
135. Jiao, J. G., et al., A plasmid DNA vaccine encoding the extracellular domain of porcine endoglin induces anti-tumour immune response against self-endoglin-related angiogenesis in two liver cancer models. Dig Liver Dis, 2006. 38(8): p. 578-87.
136. Tanaka, M., et al., Human umbilical vein endothelial cell vaccine therapy in patients with recurrent glioblastoma. Cancer Sci, 2013. 104(2): p. 200-5.
137. Zhou, L., et al., Assessment of in vivo anti-tumor activity of human umbilical vein endothelial cell vaccines prepared by various antigenforms. Eur J Pharm Sci, 2017. 114: p. 228-237.
138. Ramsey, D. E., et al., Chemoembolization of hepatocellular carcinoma. J Vasc Interv Radiol, 2002. 13(9 Pt 2): p. S211-21.
139. Sigurdson, E. R., et al., Tumor and liver drug uptake following hepatic artery and portal vein infusion. J Clin Oncol, 1987. 5(11): p. 1836-40.
140. Breedis, C. and G. Young, The blood supply of neoplasms in the liver. Am J Pathol, 1954. 30(5): p. 969-77.
141. McDermott, W. V., Jr., et al., Dearterialization of the liver for metastatic cancer. Clinical, angiographic and pathologic observations. Ann Surg, 1978. 187(1): p. 38-46.
142. Clouse, M. E., et al., Hepatic artery embolization for metastatic endocrine-secreting tumors of the pancreas. Report of two cases. Gastroenterology, 1983. 85(5): p. 1183-6.
143. Nakao, N., et al., Hepatocellular carcinoma: combined hepatic, arterial, and portal venous embolization. Radiology, 1986. 161(2): p. 303-7.
144. Hwang, T. L., et al., Resection of hepatocellular carcinoma after transcatheter arterial embolization. Reevaluation of the advantages and disadvantages of preoperative embolization. Arch Surg, 1987. 122(7): p. 756-9.
145. Stuart, K., Chemoembolization in the management of liver tumors. Oncologist, 2003. 8(5): p. 425-37.
146. Pulendran, B., Immune activation: death, danger and dendritic cells. Curr Biol, 2004. 14(1): p. R30-2.
147. Rock, K. L., et al., Natural endogenous adjuvants. Springer Semin Immunopathol, 2005. 26(3): p. 231-46.
148. McBride, W. H., et al., A sense of danger from radiation. Radiat Res, 2004. 162(1): p. 1-19.
149. Friedman, E. J., Immune modulation by ionizing radiation and its implications for cancer immunotherapy. Curr Pharm Des, 2002. 8(19): p. 1765-80.
150. Sauter, B., et al., Consequences of cell death: exposure to necrotic tumor cells, but not primary tissue cells or apoptotic cells, induces the maturation of immunostimulatory dendritic cells. J Exp Med, 2000. 191(3): p. 423-34.
151. Bzowska, M., et al., Increased IL-10 production during spontaneous apoptosis of monocytes. Eur J Immunol, 2002. 32(7): p. 2011-20.
152. Cvetanovic, M. and D. S. Ucker, Innate immune discrimination of apoptotic cells: repression of proinflammatory macrophage transcription is coupled directly to specific recognition. J Immunol, 2004. 172(2): p. 880-9.
153. Hofffmann, P. R., et al., Interaction between phosphatidylserine and the phosphatidylserine receptor inhibits immune responses in vivo. J Immunol, 2005. 174(3): p. 1393-404.
154. Reiter, I., B. Krammer, and G. Schwamberger, Cutting edge: differential effect of apoptotic versus necrotic tumor cells on macrophage antitumor activities. J Immunol, 1999. 163(4): p. 1730-2.
155. Torre-Amione, G., et al., Effects of a novel immune modulation therapy in patients with advanced chronic heart failure: results of a randomized, controlled, phase II trial. J Am Coll Cardiol, 2004. 44(6): p. 1181-6.
156. Basu, S., et al., Necrotic but not apoptotic cell death releases heat shock proteins, which deliver a partial maturation signal to dendritic cells and activate the NF-kappa B pathway. Int Immunol, 2000. 12(11): p. 1539-46.
157. Quintana, F. J. and I. R. Cohen, Heat shock proteins as endogenous adjuvants in sterile and septic inflammation. J Immunol, 2005. 175(5): p. 2777-82.
158. Tsan, M. F. and B. Gao, Endogenous ligands of Toll-like receptors. J Leukoc Biol, 2004. 76(3): p. 514-9.
159. Rovere-Querini, P., et al., HIGB1 is an endogenous immune adjuvant released by necrotic cells. EMBO Rep, 2004. 5(8): p. 825-30.
160. Kariko, K., et al., mRNA is an endogenous ligand for Toll-like receptor 3. J Biol Chem, 2004. 279(13): p. 12542-50.
161. Barrat, F. J., et al., Nucleic acids of mammalian origin can act as endogenous ligands for Toll-like receptors and may promote systemic lupus erythematosus. J Exp Med, 2005. 202(8): p. 1131-9.
162. Christensen, S. R., et al., Toll-like receptor 9 controls anti-DNA autoantibody production in murine lupus. J Exp Med, 2005. 202(2): p. 321-31.
163. Wu, F., et al., Activated anti-tumor immunity in cancer patients after high intensity focused ultrasound ablation. Ultrasound Med Biol, 2004. 30(9): p. 1217-22.
164. Ormandy, L. A., et al., Increased populations of regulatory T cells in peripheral blood of patients with hepatocellular carcinoma. Cancer Res, 2005. 65(6): p. 2457-64.
165. Jessup, J. M., et al., Carcinoembryonic antigen promotes tumor cell survival in liver through an IL-10-dependent pathway. Clin Exp Metastasis, 2004. 21(8): p. 709-17.
166. Yuan, L., et al., Restoration of macrophage tumoricidal activity by bleomycin correlates with the decreased production of transforming growth factor beta in rats bearing KDH-8 hepatoma cells. Cancer Immunol Immunother, 1997. 45(2): p. 71-6.
167. Sondak, V. K., et al., Suppressive effects of visceral tumor on the generation of antitumor T cells for adoptive immunotherapy. Arch Surg, 1991. 126(4): p. 442-6.
168. Griffini, P., et al., Kupffer cells and pit cells are not effective in the defense against experimentally induced colon carcinoma metastasis in rat liver. Clin Exp Metastasis, 1996. 14(4): p. 367-80.
169. Crispe, I. N., et al., The liver as a site of T-cell apoptosis: graveyard, or killing field? Immunol Rev, 2000. 174: p. 47-62.
170. Kara, E., et al., Effect of portal venous injection of donor spleen cells on skin allograft survival in rat. Indian J Med Res, 2004. 119(3): p. 110-4.
171. Yu, S., Y. Nakafusa, and M. W. Flye, Portal vein administration of donor cells promotes peripheral allospecific hyporesponsiveness and graft tolerance. Surgery, 1994. 116(2): p. 229-34; discussion 234-5.
172. Hamashima, T., et al., [Effects of portal venous administration with allogenic cells on renal allograft survival in the rat]. Nippon Geka Gakkai Zasshi, 1989. 90(10): p. 1752-7.
173. Nakano, Y., et al., Permanent acceptance of liver allografts by intraportal injection of donor spleen cells in rats. Surgery, 1992. 111(6): p. 668-76.
174. Carr, R. I., et al., Induction of transplantation tolerance by feeding or portal vein injection pretreatment of recipient with donor cells. Ann N Y Acad Sci, 1996. 778: p. 368-70.
175. Asakura, H., et al., The persistence of regulatory cells developing after rat spontaneous liver acceptance. Surgery, 2005. 138(2): p. 329-34.
176. Reding, R. and H. F. Davies, Revisiting liver transplant immunology: from the concept of immune engagement to the dualistic pathway paradigm. Liver Transpl, 2004. 10(9): p. 1081-6.
177. Delriviere, L., et al., Administration of exogenous interleukin-2 abrogates spontaneous rat liver allograft acceptance but does not affect long-term established graft survival. Transplantation, 1997. 63(11): p. 1698-701.
178. den Dulk, M. and G. A. Bishop, Immune mechanisms contributing to spontaneous acceptance of liver transplants in rodents and their potential for clinical transplantation. Arch Immunol Ther Exp (Warsz), 2003. 51(1): p. 29-44.
179. Steptoe, R. J., et al., Augmentation of dendritic cells in murine organ donors by Flt3 ligand alters the balance between transplant tolerance and immunity. J Immunol, 1997. 159(11): p. 5483-91.
180. Limmer, A., et al., Cross-presentation of oral antigens by liver sinusoidal endothelial cells leads to CD8 T cell tolerance. Eur J Immunol, 2005. 35(10): p. 2970-81.
181. Onoe, T., et al., Liver sinusoidal endothelial cells tolerize T cells across MHC barriers in mice. J Immunol, 2005. 175(1): p. 139-46.
182. Crispe, I. N., Hepatic T cells and liver tolerance. Nat Rev Immunol, 2003. 3(1): p. 51-62.
183. Sharif, S., et al., Activation of natural killer T cells by alpha-galactosylceramide treatment prevents the onset and recurrence of autoimmune Type 1 diabetes. Nat Med, 2001. 7(9): p. 1057-62.
184. Prehn, R. T., Proceedings: Immune involvement in oncogenesis. Proc Natl Cancer Conf, 1972. 7: p. 401-4.
185. Shah, S. R., et al., Tumour ablation and hepatic decompensation rates in multi-agent chemoembolization of hepatocellular carcinoma. Qjm, 1998. 91(12): p. 821-8.
186. Donepudi, M., et al., Mechanism of melphalan-induced B7-1 gene expression in P815 tumor cells. J Immunol, 2001. 166(11): p. 6491-9.
187. Donepudi, M., et al., Melphalan-induced up-regulation of B7-1 surface expression on normal splenic B cells. Cancer Immunol Immunother, 2003. 52(3): p. 162-70.
188. Ng, C. S., et al., Mechanisms of immune evasion by renal cell carcinoma: tumor-induced T-lymphocyte apoptosis and NFkappaB suppression. Urology, 2002. 59(1): p. 9-14.
189. Campbell, J. D., et al., Suppression of IL-2-induced T cell proliferation and
phosphorylation of STAT3 and STATS by tumor-derived TGF beta is reversed by IL-15. J Immunol, 2001. 167(1): p. 553-61.
190. Beck, C., H. Schreiber, and D. Rowley, Role of TGF-beta in immune-evasion of cancer. Microsc Res Tech, 2001. 52(4): p. 387-95.
191. Almand, B., et al., Increased production of immature myeloid cells in cancer patients: a mechanism ofimmunosuppression in cancer. J Immunol, 2001. 166(1): p. 678-89.
192. Dix, A. R., et al., Immune defects observed inpatients with primary malignant brain tumors. J Neuroimmunol, 1999. 100(1-2): p. 216-32.
193. Kiessling, R., et al., Tumor-induced immune dysfunction. Cancer Immunol Immunother, 1999. 48(7): p. 353-62.
194. Kim, H. J., J. K. Park, and Y. G. Kim, Suppression of NF-kappaB activation in normal T cells by supernatant fluid from human renal cell carcinomas. J Korean Med Sci, 1999. 14(3): p. 299-303.
195. Ungefroren, H., et al., Immunological escape mechanisms in pancreatic carcinoma. Ann N Y Acad Sci, 1999. 880: p. 243-51.
196. Fischer, J. R., et al., Decrease of interleukin-2 secretion is a new independent prognostic factor associated with poor survival inpatients with small-cell lung cancer. Ann Oncol, 1997. 8(5): p. 457-61.
197. Ishigami, S., et al., CD3-zetachain expression of intratumoral lymphocytes is closely related to survival in gastric carcinoma patients. Cancer, 2002. 94(5): p. 1437-42.
198. Marana, H. R., et al., Reduced immunologic cell performance as a prognostic parameter for advanced cervical cancer. Int J Gynecol Cancer, 2000. 10(1): p. 67-73.
199. Gastman, B. R., et al., Tumor-induced apoptosis of T lymphocytes: elucidation of intracellular apoptotic events. Blood, 2000. 95(6): p. 2015-23.
200. Takahashi, A., et al., Elevated caspase-3 activity in peripheral blood T cells coexists with increased degree of T-cell apoptosis and down-regulation of TCR zeta molecules in patients with gastric cancer. Clin Cancer Res, 2001. 7(1): p. 74-80.
201. Mizoguchi, H., et al., Alterations in signal transduction molecules in T lymphocytes from tumor-bearing mice. Science, 1992. 258(5089): p. 1795-8.
202. Horiguchi, S., et al., Primary chemically induced tumors induce profound immunosuppression concomitant with apoptosis and alterations in signal transduction in T cells and NK cells. Cancer Res, 1999. 59(12): p. 2950-6.
203. Schmielau, J. and O. J. Finn, Activated granulocytes and granulocyte-derived hydrogen peroxide are the underlying mechanism of suppression of t-cell function in advanced cancer patients. Cancer Res, 2001. 61(12): p. 4756-60.
204. Kim, C. W., et al., Alteration of signal-transducing molecules and phenotypical characteristics in peripheral blood lymphocytes from gastric carcinoma patients. Pathobiology, 1999. 67(3): p. 123-8.
205. Laytragoon-Lewin, N., et al., Alteration of cellular mediated cytotoxicity, T cell receptor zeta (TcR zeta) and apoptosis related gene expression in nasopharyngeal carcinoma (NPC) patients: possible clinical relevance. Anticancer Res, 2000. 20(2B): p. 1093-100.
206. Taylor, D. D., et al., Modulation of TcR/CD3-zeta chain expression by a circulating factor derived from ovarian cancer patients. Br J Cancer, 2001. 84(12): p. 1624-9.
207. Chen, X., et al., Impaired expression of the CD3-zeta chain in peripheral blood T cells of patients with chronic myeloid leukaemia results in an increased susceptibility to apoptosis. Br J Haematol, 2000. 111(3): p. 817-25.
208. Healy, C. G., et al., Impaired expression and function of signal-transducing zeta chains in peripheral T cells and natural killer cells in patients with prostate cancer. Cytometry, 1998. 32(2): p. 109-19.
209. Valkovic, T., et al., Correlation between vascular endothelial growth factor, angiogenesis, and tumor-associated macrophages in invasive ductal breast carcinoma. Virchows Arch, 2002. 440(6): p. 583-8.
210. Makitie, T., et al., Tumor-infiltrating macrophages (CD68(+) cells) and prognosis in malignant uveal melanoma. Invest Ophthalmol Vis Sci, 2001. 42(7): p. 1414-21.
211. Leek, R. D., et al., Association of macrophage infiltration with angiogenesis and prognosis in invasive breast carcinoma. Cancer Res, 1996. 56(20): p. 4625-9.
212. Lewis, J. S., et al., Expression of vascular endothelial growth factor by macrophages is up-regulated in poorly vascularized areas of breast carcinomas. J Pathol, 2000. 192(2): p. 150-8.
213. Nowicki, A., et al., Impaired tumor growth in colony-stimulating factor 1 (CSF-1)-deficient, macrophage-deficient op/op mouse: evidence for a role of CSF-1-dependent macrophages information of tumor stroma. Int J Cancer, 1996. 65(1): p. 112-9.
214. Kamate, C., et al., Inflammation and cancer, the mastocytoma P815 tumor model revisited: triggering of macrophage activation in vivo with pro-tumorigenic consequences. Int J Cancer, 2002. 100(5): p. 571-9.
215. Young, M. R., et al., Suppressor alveolar macrophages in mice bearing metastatic Lewis lung carcinoma tumors. J Leukoc Biol, 1987. 42(6): p. 682-8.
216. Billingsley, K. G., et al., Macrophage-derived tumor necrosis factor and tumor-derived of leukemia inhibitory factor and interleukin-6: possible cellular mechanisms of cancer cachexia. Ann Surg Oncol, 1996. 3(1): p. 29-35.
217. Bonta, I. L. and S. Ben-Efraim, Involvement of inflammatory mediators in macrophage antitumor activity. J Leukoc Biol, 1993. 54(6): p. 613-26.
218. Bhaumik, S. and A. Khar, Induction of nitric oxide production by the peritoneal macrophages after intraperitoneal or subcutaneous transplantation of AK-5 tumor. Nitric Oxide, 1998. 2(6): p. 467-74.
219. Lewis, J. G. and D. O. Adams, Inflammation, oxidative DNA damage, and carcinogenesis. Environ Health Perspect, 1987. 76: p. 19-27.
220. Kono, K., et al., Hydrogen peroxide secreted by tumor-derived macrophages down-modulates signal-transducing zeta molecules and inhibits tumor-specific T cell-and natural killer cell-mediated cytotoxicity. Eur J Immunol, 1996. 26(6): p. 1308-13.
221. Murr, C., et al., Neopterin as a marker for immune system activation. Curr Drug Metab, 2002. 3(2): p. 175-87.
222. Whisler, R. L., L. S. Gray, and K. V. Hackshaw, Rheumatology, a clinical overview. Clin Podiatr Med Surg, 2002. 19(1): p. 149-61, vii.
223. Ishihara, K. and T. Hirano, IL-6 in autoimmune disease and chronic inflammatory proliferative disease. Cytokine Growth Factor Rev, 2002. 13(4-5): p. 357.
224. Mahmoud, F. A. and N. I. Rivera, The role of C-reactive protein as a prognostic indicator in advanced cancer. Curr Oncol Rep, 2002. 4(3): p. 250-5.
225. Smith, P. C., et al., Interleukin-6 and prostate cancer progression. Cytokine Growth Factor Rev, 2001. 12(1): p. 33-40.
226. Rutkowski, P., et al., Cytokine serum levels in soft tissue sarcoma patients: correlations with clinico-pathological features and prognosis. Int J Cancer, 2002. 100(4): p. 463-71.
227. Kallio, J. P., et al., Soluble immunological parameters and early prognosis of renal cell cancer patients. J Exp Clin Cancer Res, 2001. 20(4): p. 523-8.
228. Ljungberg, B., K. Grankvist, and T. Rasmuson, Serum interleukin-6 in relation to acute-phase reactants and survival inpatients with renal cell carcinoma. Eur J Cancer, 1997. 33(11): p. 1794-8.
229. Fearon, K. C., et al., Pancreatic cancer as a model: inflammatory mediators, acute-phase response, and cancer cachexia. World J Surg, 1999. 23(6): p. 584-8.
230. McMillan, D. C., et al., Albumin concentrations are primarily determined by the body cell mass and the systemic inflammatory response in cancer patients with weight loss. Nutr Cancer, 2001. 39(2): p. 210-3.
231. Oya, M., et al., High preoperative plasma D-dimer level is associated with advanced tumor stage and short survival after curative resection in patients with colorectal cancer. Jpn J Clin Oncol, 2001. 31(8): p. 388-94.
232. Ferrigno, D., G. Buccheri, and I. Ricca, Prognostic significance of blood coagulation tests in lung cancer. Eur Respir J, 2001. 17(4): p. 667-73.
233. Blackwell, K., et al., Plasma D-dimer levels in operable breast cancer patients correlate with clinical stage and axillary lymph node status. J Clin Oncol, 2000. 18(3): p. 600-8.
234. Tabuchi, T., et al., Granulocyte apheresis as a possible new approach in cancer therapy: A pilot study involving two cases. Cancer Detect Prev, 1999. 23(5): p. 417-21.
235. Oberholzer, A., C. Oberholzer, and L. L. Moldawer, Sepsis syndromes: understanding the role of innate and acquired immunity. Shock, 2001. 16(2): p. 83-96.
236. Heidecke, C. D., et al., Selective defects of T lymphocyte function inpatients with lethal intraabdominal infection. Am J Surg, 1999. 178(4): p. 288-92.
237. Elgert, K. D., D. G. Alleva, and D. W. Mullins, Tumor-induced immune dysfunction: the macrophage connection. J Leukoc Biol, 1998. 64(3): p. 275-90.
238. De la Fuente, M. and V. M. Victor, Ascorbic acid and N-acetylcysteine improve in vitro the function of lymphocytes from mice with endotoxin-induced oxidative stress. Free Radic Res, 2001. 35(1): p. 73-84.
239. Malmberg, K. J., et al., A short-term dietary supplementation of high doses of vitamin E increases T helper 1 cytokine production inpatients with advanced colorectal cancer. Clin Cancer Res, 2002. 8(6): p. 1772-8.
240. Heller, A. R., et al., N-acetylcysteine reduces respiratory burst but augments neutrophil phagocytosis in intensive care unit patients. Crit Care Med, 2001. 29(2): p. 272-6.
241. Van Schooten, F. J., et al., Effects of oral administration of N-acetyl-L-cysteine: a multi-biomarker study in smokers. Cancer Epidemiol Biomarkers Prev, 2002. 11(2): p. 167-75.
242. Albini, A., et al., Inhibition of angiogenesis-driven Kaposi's sarcoma tumor growth in nude mice by oral N-acetylcysteine. Cancer Res, 2001. 61(22): p. 8171-8.
243. Leibovitz, B. and B. V. Siegel, Ascorbic acid, neutrophil function, and the immune response. Int J Vitam Nutr Res, 1978. 48(2): p. 159-64.
244. Siegel, B. V., Enhanced interferon response to murine leukemia virus by ascorbic acid. Infect Immun, 1974. 10(2): p. 409-10.
245. Riordan, H. D., et al., Intravenous ascorbic acid: protocol for its application and use. P R Health Sci J, 2003. 22(3): p. 287-90.
246. Folkers, K. and A. Wolaniuk, Research on coenzyme Q1O in clinical medicine and in immunomodulation. Drugs Exp Clin Res, 1985. 11(8): p. 539-45.
247. Ravindranath, M. H., et al., Anticancer therapeutic potential of soy isoflavone, genistein. Adv Exp Med Biol, 2004. 546: p. 121-65.
248. Li, T., et al., [Preliminary study on anti-tumor function of resveratrol and its immunological mechanism.]. Xi Bao Yu Fen Zi Mian Yi Xue Za Zhi, 2005. 21(5): p. 575-9.
249. Nestle, F. O., et al., Vaccination of melanoma patients with peptide-or tumor lysate-pulsed dendritic cells. Nat Med, 1998. 4(3): p. 328-32.
250. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
251. Wang, F., et al., Phase I trial of a MART-1 peptide vaccine with incomplete Freund's adjuvant for resected high-risk melanoma. Clin Cancer Res, 1999. 5(10): p. 2756-65.
252. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
253. Thomas, R., et al., Immature human monocyte-derived dendritic cells migrate rapidly to draining lymph nodes after intradermal injection for melanoma immunotherapy. Melanoma Res, 1999. 9(5): p. 474-81.
254. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
255. Panelli, M. C., et al., Phase 1 study inpatients with metastatic melanoma of immunization with dendritic cells presenting epitopes derived from the melanoma-associated antigens MART-1 and gp100. J Immunother, 2000. 23(4): p. 487-98.
256. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
257. Lau, R., et al., Phase I trial of intravenous peptide-pulsed dendritic cells in patients with metastatic melanoma. J Immunother, 2001. 24(1): p. 66-78.
258. Banchereau, J., et al., Immune and clinical responses inpatients with metastatic melanoma to CD34(+) progenitor-derived dendritic cell vaccine. Cancer Res, 2001. 61(17): p. 6451-8.
259. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
260. Palucka, A. K., et al., Single injection of CD34+ progenitor-derived dendritic cell vaccine can lead to induction of T-cell immunity inpatients with stage IV melanoma. J Immunother, 2003. 26(5): p. 432-9.
261. Bedrosian, I., et al., Intranodal administration of peptide-pulsed mature dendritic cell vaccines results in superior CD8+T-cell function in melanoma patients. J Clin Oncol, 2003. 21(20): p. 3826-35.
262. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
263. Hersey, P., et al., Phase I/II study of treatment with dendritic cell vaccines in patients with disseminated melanoma. Cancer Immunol Immunother, 2004. 53(2): p. 125-34.
264. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
265. Palucka, A. K., et al., Spontaneous proliferation and type 2 cytokine secretion by CD4+ T cells inpatients with metastatic melanoma vaccinated with antigen-pulsed dendritic cells. J Clin Immunol, 2005. 25(3): p. 288-95.
266. Banchereau, J., et al., Immune and clinical outcomes inpatients with stage IV melanoma vaccinated with peptide-pulsed dendritic cells derived from CD34+ progenitors and activated with type I interferon. J Immunother, 2005. 28(5): p. 505-16.
267. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
268. Salcedo, M., et al., Vaccination of melanoma patients using dendritic cells loaded with an allogeneic tumor cell lysate. Cancer Immunol Immunother, 2006. 55(7): p. 819-29.
269. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
270. Escobar, A., et al., Dendritic cell immunizations alone or combined with low doses of interleukin-2 induce specific immune responses in melanoma patients. Clin Exp Immunol, 2005. 142(3): p. 555-68.
271. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
272. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.
273. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
274. Nakai, N., et al., Vaccination of Japanese patients with advanced melanoma with peptide, tumor lysate or both peptide and tumor lysate-pulsed mature, monocyte-derived dendritic cells. J Dermatol, 2006. 33(7): p. 462-72.
275. Palucka, A. K., et al., Dendritic cells loaded with killed allogeneic melanoma cells can induce objective clinical responses and MART-1 specific CD8+T-cell immunity. J Immunother, 2006. 29(5): p. 545-57.
276. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
277. Guo, J., et al., Intratumoral injection of dendritic cells in combination with local hyperthermia induces systemic antitumor effect inpatients with advanced melanoma. Int J Cancer, 2007. 120(11): p. 2418-25.
278. O'Rourke, M. G., et al., Dendritic cell immunotherapy for stage IV melanoma. Melanoma Res, 2007. 17(5): p. 316-22.
279. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
280. Hersey, P., et al., Phase I/I study of treatment with matured dendritic cells with or without low dose IL-2 inpatients with disseminated melanoma. Cancer Immunol Immunother, 2008. 57(7): p. 1039-51.
281. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
282. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
283. Redman, B. G., et al., Phase Ib trial assessing autologous, tumor-pulsed dendritic cells as a vaccine administered with or without IL-2 inpatients with metastatic melanoma. J Immunother, 2008. 31(6): p. 591-8.
284. Daud, A. I., et al., Phenotypic and functional analysis of dendritic cells and clinical outcome inpatients with high-risk melanoma treated with adjuvant granulocyte macrophage colony-stimulating factor. J Clin Oncol, 2008. 26(19): p. 3235-41.
285. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
286. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
287. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
288. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
289. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
290. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
291. Ribas, A., et al., Multicenter phase II study of matured dendritic cells pulsed with melanoma cell line lysates inpatients with advanced melanoma. J Transl Med, 2010. 8: p. 89.
292. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
293. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
294. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.
295. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
296. Steele, J. C., et al., Phase I/II trial of a dendritic cell vaccine transfected with DNA encoding melan A and gp100 for patients with metastatic melanoma. Gene Ther, 2011. 18(6): p. 584-93.
297. Kim, D. S., et al., Immunotherapy of malignant melanoma with tumor lysate-pulsed autologous monocyte-derived dendritic cells. Yonsei Med J, 2011. 52(6): p. 990-8.
298. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
299. Dillman, R. O., et al., Tumor stem cell antigens as consolidative active specific immunotherapy: a randomized phase II trial of dendritic cells versus tumor cells inpatients with metastatic melanoma. J Immunother, 2012. 35(8): p. 641-9.
300. Dannull, J., et al., Melanoma immunotherapy using mature DCs expressing the constitutive proteasome. J Clin Invest, 2013. 123(7): p. 3135-45.
301. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
302. Stift, A., et al., Dendritic cell vaccination in medullary thyroid carcinoma. Clin Cancer Res, 2004. 10(9): p. 2944-53.
303. Kuwabara, K., et al., Results of a phase I clinical study using dendritic cell vaccinations for thyroid cancer. Thyroid, 2007. 17(1): p. 53-8.
304. Bachleitner-Hofmann, T., et al., Pilot trial of autologous dendritic cells loaded with tumor lysate(s) from allogeneic tumor cell lines inpatients with metastatic medullary thyroid carcinoma. Oncol Rep, 2009. 21(6): p. 1585-92.
305. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.
306. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
307. Yu, J. S., et al., Vaccination with tumor lysate-pulsed dendritic cells elicits antigen-specific, cytotoxic T-cells inpatients with malignant glioma. Cancer Res, 2004. 64(14): p. 4973-9.
308. Yamanaka, R., et al., Tumor lysate and IL-18 loaded dendritic cells elicits Th1 response, tumor-specific CD8+ cytotoxic T cells inpatients with malignant glioma. J Neurooncol, 2005. 72(2): p. 107-13.
309. Yamanaka, R., et al., Clinical evaluation of dendritic cell vaccination for patients with recurrent glioma: results of a clinical phase I/II trial. Clin Cancer Res, 2005. 11(11): p. 4160-7.
310. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
311. Walker, D. G., et al., Results of a phase I dendritic cell vaccine trial for malignant astrocytoma: potential interaction with adjuvant chemotherapy. J Clin Neurosci, 2008. 15(2): p. 114-21.
312. Leplina, O. Y., et al., Use of interferon-alpha-induced dendritic cells in the therapy of patients with malignant brain gliomas. Bull Exp Biol Med, 2007. 143(4): p. 528-34.
313. De Vleeschouwer, S., et al., Postoperative adjuvant dendritic cell-based immunotherapy inpatients with relapsed glioblastoma multiforme. Clin Cancer Res, 2008. 14(10): p. 3098-104.
314. Ardon, H., et al., Adjuvant dendritic cell-based tumour vaccination for children with malignant brain tumours. Pediatr Blood Cancer, 2010. 54(4): p. 519-25.
315. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
316. Okada, H., et al., Induction of CD8+T-cell responses against novel glioma-associated antigen peptides and clinical activity by vaccinations with {alpha}-type 1 polarized dendritic cells and polyinosinic-polycytidylic acid stabilized by lysine and carboxymethylcellulose inpatients with recurrent malignant glioma. J Clin Oncol, 2011. 29(3): p. 330-6.
317. Fadul, C. E., et al., Immune response inpatients with newly diagnosed glioblastoma multiforme treated with intranodal autologous tumor lysate-dendritic cell vaccination after radiation chemotherapy. J Immunother, 2011. 34(4): p. 382-9.
318. Chang, C. N., et al., A phase I/II clinical trial investigating the adverse and therapeutic effects of a postoperative autologous dendritic cell tumor vaccine inpatients with malignant glioma. J Clin Neurosci, 2011. 18(8): p. 1048-54.
319. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
320. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.
321. Fong, B., et al., Monitoring of regulatory T cell frequencies and expression of CTLA-4 on T cells, before and after DC vaccination, can predict survival in GBM patients. PLoS One, 2012. 7(4): p. e32614.
322. De Vleeschouwer, S., et al., Stratification according to HGG-MMIUNO RPA model predicts outcome in a large group of patients with relapsed malignant glioma treated by adjuvant postoperative dendritic cell vaccination. Cancer Immunol Immunother, 2012. 61(11): p. 2105-12.
323. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
324. Akiyama, Y., et al., alpha-type-1 polarized dendritic cell-based vaccination in recurrent high-grade glioma: a phase I clinical trial. BMC Cancer, 2012. 12: p. 623.
325. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
326. Shah, A. H., et al., Dendritic cell vaccine for recurrent high-grade gliomas in pediatric and adult subjects: clinical trial protocol. Neurosurgery, 2013. 73(5): p. 863-7.
327. Reichardt, V. L., et al., Idiotype vaccination using dendritic cells after autologous peripheral blood stem cell transplantation for multiple myeloma-a feasibility study. Blood, 1999. 93(7): p. 2411-9.
328. Lim, S. H. and R. Bailey-Wood, Idiotypic protein-pulsed dendritic cell vaccination in multiple myeloma. Int J Cancer, 1999. 83(2): p. 215-22.
329. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
330. Reichardt, V. L., et al., Idiotype vaccination of multiple myeloma patients using monocyte-derived dendritic cells. Haematologica, 2003. 88(10): p. 1139-49.
331. Guardino, A. E., et al., Production of myeloid dendritic cells (DC) pulsed with tumor-specific idiotype protein for vaccination of patients with multiple myeloma. Cytotherapy, 2006. 8(3): p. 277-89.
332. Lacy, M. Q., et al., Idiotype-pulsed antigen-presenting cells following autologous transplantation for multiple myeloma may be associated with prolonged survival. Am J Hematol, 2009. 84(12): p. 799-802.
333. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150(5): p. 554-64.
334. Rollig, C., et al., Induction of cellular immune responses inpatients with stage-I multiple myeloma after vaccination with autologous idiotype-pulsed dendritic cells. J Immunother, 2011. 34(1): p. 100-6.
335. Zahradova, L., et al., Efficacy and safety of Id-protein-loaded dendritic cell vaccine inpatients with multiple myeloma-phase II study results. Neoplasma, 2012. 59(4): p. 440-9.
336. Timmerman, J. M., et al., Idiotype-pulsed dendritic cell vaccination for B-cell lymphoma: clinical and immune responses in 35 patients. Blood, 2002. 99(5): p. 1517-26.
337. Maier, T., et al., Vaccination of patients with cutaneous T-cell lymphoma using intranodal injection of autologous tumor-lysate-pulsed dendritic cells. Blood, 2003. 102(7): p. 2338-44.
338. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
339. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
340. Li, L., et al., Immunotherapy for patients with acute myeloid leukemia using autologous dendritic cells generated from leukemic blasts. Int J Oncol, 2006. 28(4): p. 855-61.
341. Roddie, H., et al., Phase I/II study of vaccination with dendritic-like leukaemia cells for the immunotherapy of acute myeloid leukaemia. Br J Haematol, 2006. 133(2): p. 152-7.
342. Litzow, M. R., et al., Testing the safety of clinical-grade mature autologous myeloid DC in a phase I clinical immunotherapy trial of CML. Cytotherapy, 2006. 8(3): p. 290-8.
343. Westermann, J., et al., Vaccination with autologous non-irradiated dendritic cells in patients with bcr/abl+ chronic myeloid leukaemia. Br J Haematol, 2007. 137(4): p. 297-306.
344. Hus, I., et al., Vaccination of B-CLL patients with autologous dendritic cells can change the frequency of leukemia antigen-specific CD8+ T cells as well as CD4+CD25+FoxP3+ regulatory T cells toward an antileukemia response. Leukemia, 2008. 22(5): p. 1007-17.
345. Palma, M., et al., Development of a dendritic cell-based vaccine for chronic lymphocytic leukemia. Cancer Immunol Immunother, 2008. 57(11): p. 1705-10.
346. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
347. Iwashita, Y., et al., A phase I study of autologous dendritic cell-based immunotherapy for patients with unresectable primary liver cancer. Cancer Immunol Immunother, 2003. 52(3): p. 155-61.
348. Lee, W. C., et al., Vaccination of advanced hepatocellular carcinoma patients with tumor lysate-pulsed dendritic cells: a clinical trial. J Immunother, 2005. 28(5): p. 496-504.
349. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
350. Palmer, D. H., et al., A phase II study of adoptive immunotherapy using dendritic cells pulsed with tumor lysate inpatients with hepatocellular carcinoma. Hepatology, 2009. 49(1): p. 124-32.
351. El Ansary, M., et al., Immunotherapy by autologous dendritic cell vaccine in patients with advanced HCC. J Cancer Res Clin Oncol, 2013. 139(1): p. 39-48.
352. Tada, F., et al., Phase I/II study of immunotherapy using tumor antigen-pulsed dendritic cells inpatients with hepatocellular carcinoma. Int J Oncol, 2012. 41(5): p. 1601-9.
353. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
354. Hirschowitz, E. A., et al., Autologous dendritic cell vaccines for non-small-cell lung cancer. J Clin Oncol, 2004. 22(14): p. 2808-15.
355. Chang, G. C., et al., A pilot clinical trial of vaccination with dendritic cells pulsed with autologous tumor cells derived from malignant pleural effusion inpatients with late-stage lung carcinoma. Cancer, 2005. 103(4): p. 763-71.
356. Yannelli, J. R., et al., The large scale generation of dendritic cells for the immunization of patients with non-small cell lung cancer (NSCLC). Lung Cancer, 2005. 47(3): p. 337-50.
357. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
358. Antonia, S. J., et al., Combination of p53 cancer vaccine with chemotherapy in patients with extensive stage small cell lung cancer. Clin Cancer Res, 2006. 12(3 Pt 1): p. 878-87.
359. Perrot, I., et al., Dendritic cells infiltrating human non-small cell lung cancer are blocked at immature stage. J Immunol, 2007. 178(5): p. 2763-9.
360. Hirschowitz, E. A., et al., Immunization of NSCLC patients with antigen-pulsed immature autologous dendritic cells. Lung Cancer, 2007. 57(3): p. 365-72.
361. Baratelli, F., et al., Pre-clinical characterization of GMP grade CCL21-gene modified dendritic cells for application in a phase I trial in non-small cell lung cancer. J Transl Med, 2008. 6: p. 38.
362. Hegmans, J. P., et al., Consolidative dendritic cell-based immunotherapy elicits cytotoxicity against malignant mesothelioma. Am J Respir Crit Care Med, 2010. 181(12): p. 1383-90.
363. Um, S. J., et al., Phase I study of autologous dendritic cell tumor vaccine in patients with non-small cell lung cancer. Lung Cancer, 2010. 70(2): p. 188-94.
364. Chiappori, A. A., et al., INGN-225: a dendritic cell-based p53 vaccine (Adp53-DC) in small cell lung cancer: observed association between immune response and enhanced chemotherapy effect. Expert Opin Biol Ther, 2010. 10(6): p. 983-91.
365. Perroud, M. W., Jr., et al., Mature autologous dendritic cell vaccines in advanced non-small cell lung cancer: a phase I pilot study. J Exp Clin Cancer Res, 2011. 30: p. 65.
366. Skachkova, O. V., et al., Immunological markers of anti-tumor dendritic cells vaccine efficiency inpatients with non-small cell lung cancer. Exp Oncol, 2013. 35(2): p. 109-13.
367. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51(1): p. 45-52.
368. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
369. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
370. Kandalaft, L. E., et al., A Phase I vaccine trial using dendritic cells pulsed with autologous oxidized lysatefor recurrent ovarian cancer. J Transl Med, 2013. 11: p. 149.
371. Lepisto, A. J., et al., A phase I/II study of a MUCI peptide pulsed autologous dendritic cell vaccine as adjuvant therapy inpatients with resected pancreatic and biliary tumors. Cancer Ther, 2008. 6(B): p. 955-964.
372. Rong, Y., et al., A phase I pilot trial of MUC-peptide-pulsed dendritic cells in the treatment of advanced pancreatic cancer. Clin Exp Med, 2012. 12(3): p. 173-80.
373. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method of inducing cell death of tumor cells in an individual, comprising the step of administering to the individual a therapeutically effective amount of a plurality of modified fibroblasts, wherein:
(I) the fibroblasts express recombinant:
(a) one or more endothelial-inducing genes and/or one or more vascular channel-inducing genes; and
(b) one or more suicide or death-inducing genes; and
(c) optionally one or more immune stimulatory genes; and/or
(II) the fibroblasts are cultured in endothelial progenitor cell conditioned media.

2. The method of claim 1, wherein said fibroblasts express recombinant ETV2, FOXC2, and/or FLI1.

3. The method of claim 1, wherein said fibroblasts express recombinant ETV2, FOXC2, and/or FLI1 and are cultured with media that comprises an effective amount of one or more of VEGF, EGF, HGF, and IGF-1.

4. (canceled)

5. (canceled)

6. The method of claim 1, wherein said endothelial cell progenitor cell conditioned media is generated from pluripotent stem cells differentiated into endothelial progenitor cells.

7. The method of claim 6, wherein said pluripotent stem cells are embryonic stem cells, inducible pluripotent stem cells, somatic nuclear transfer derived stem cells, or parthenogenically derived stem cells.

8. (canceled)

9. (canceled)

10. (canceled)

11. The method of claim 6, wherein said pluripotent stem cells are differentiated into endothelial progenitor cells by transfection of ETV2, FOXC2, and FLI1.

12. The method of claim 1, wherein said fibroblasts are transfected with one or more thrombosis-associated genes, wherein said gene is upregulated in response to hypoxia.

13. The method of claim 12, wherein said thrombosis-associated gene is tissue factor and/or an inhibitor of Protein C.

14. (canceled)

15. The method of claim 1, wherein said fibroblasts are transfected with one or more immune stimulatory genes.

16. The method of claim 15, wherein said immune stimulatory gene is inducible by the presence of hypoxia.

17. The method of claim 16, wherein induction of said immune stimulatory gene is performed by placing said gene under control of the HIF-1 alpha transcription factor.

18. The method of claim 1, wherein said immune stimulatory gene is associated with antigen presentation.

19. The method of claim 18, wherein said gene associated with antigen presentation is an allogeneic MHC molecule, xenogeneic MHC molecule and/or one or more of HLA B7 molecule, CD80, CD86, and CD40.

20. (canceled)

21. (canceled)

22. The method of claim 1, wherein said immune stimulatory molecule is interleukin-12.

23. The method of claim 1, wherein said fibroblasts are selected for expression of one or more of CXCR4, CD73, CD74, CD206, and interleukin-3 receptor.

24. A method for inducing immunogenic cell death of tumor endothelial cells in an individual, comprising the steps of:
a) transfecting a fibroblast population with one or more endothelial cell-inducing genes and/or one or more vascular channel-inducing genes;
c) transfecting said fibroblasts with one or more suicide or death-inducing genes;
d) optionally transfecting said fibroblasts with one or more immune stimulatory genes; and
e) administering said fibroblasts into an individual with cancer.

25. The method of claim 24, wherein said fibroblasts are obtained from tissues selected from the group consisting of a) dermal; b) bone marrow; c) blood; d) mobilized peripheral blood; e) gingiva; f) tonsil; g) placenta; h) Wharton's Jelly; i) hair follicle; j) fallopian tube; k) liver; l) deciduous tooth; m) vas deferens; n) endometrial; o) menstrual blood; and p) omentum.

26. The method of claim 25, wherein said mobilization of peripheral blood is achieved through treatment of a mammal with an effective amount of one or more inhibitors of SDF-1 binding to CXCR4.

27. The method of claim 26, wherein said inhibitor of SDF-1 binding to CXCR4 is Plerixafor or BKT140.

28. (canceled)

29. (canceled)

30. The method of claim 25, wherein said mobilization is induced by exposure to hyperbaric oxygen treatment, treatment with GM-CSF, treatment with M-CSF, treatment with G-CSF, or treatment with flt-3 ligand.

31. (canceled)

32. (canceled)

33. (canceled)

34. (canceled)

35. (canceled)

36. The method of claim 24, wherein said fibroblasts are selected for expression of CXCR4, CD73, CD74, CD206, or interleukin-3 receptor.

37. (canceled)

38. (canceled)

39. (canceled)

40. (canceled)

41. The method of claim 24, wherein said fibroblasts are cultured in endothelial progenitor cell-conditioned media.

42. The method of claim 41 wherein said endothelial cell progenitor cell conditioned media is generated from pluripotent stem cells differentiated into endothelial progenitor cells.

43. The method of claim 42, wherein said pluripotent stem cells are embryonic stem cells, inducible pluripotent stem cells, somatic nuclear transfer derived stem cells, or parthenogenically derived stem cells.

44. (canceled)

45. (canceled)

46. (canceled)

47. The method of claim 42, wherein said pluripotent stem cells are differentiated into endothelial progenitor cells by transfection of ETV2, FOXC2, and FLI1.

48. The method of claim 24, wherein said fibroblasts are differentiated into endothelial progenitor cells by transfection with ETV2, by transfection with ETV2 and cultured in VEGF, by transfection with ETV2 and cultured in EGF, by transfection with ETV2 and cultured in HGF, by transfection with ETV2 and cultured in IGF-1, by transfection with FOXC2, by transfection with FOXC2 and cultured in VEGF, by transfection with FOXC2 and cultured in EGF, by transfection with FOXC2 and cultured in EGF, by transfection with FOXC2 and cultured in HGF, by transfection with FOXC2 and cultured in IGF-1, by transfection with FLI1, by transfection with FLI1 and cultured in VEGF, by transfection with FLI1 and cultured in EGF, by transfection with FLI1 and cultured in HGF, and/or by transfection with FLI1 and cultured in IGF-1.

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. The method of claim 24, wherein said fibroblasts are transfected with a thrombosis associated gene, wherein said gene is upregulated in response to hypoxia.

64. The method of claim 63, wherein said thrombosis associated gene is tissue factor or is an inhibitor of Protein C.

65. (canceled)

66. The method of claim 24, wherein said fibroblast is transfected with one or more immune stimulatory genes.

67. The method of claim 66, wherein said immune stimulatory gene is inducible by the presence of hypoxia.

68. The method of claim 67, wherein induction of said immune stimulatory gene is performed by placing said gene under control of the HIF-1 alpha transcription factor.

69. The method of claim 66, wherein said immune stimulatory gene is associated with antigen presentation.

70. The method of claim 69, wherein said gene associated with antigen presentation is an allogeneic MHC molecule, a xenogeneic MHC molecule, HLA B7 molecule, CD80, CD86, or CD40.

71. (canceled)

72. (canceled)

73. (canceled)

74. (canceled)

75. (canceled)

76. The method of claim 66,
wherein said immune stimulatory gene is interleukin-12.
